(19) United States
(12) Patent Application Publication
(10) Pub. No.: US 2017/0081323 A1
(43) Pub. Date: Mar. 23, 2017

(54) Triazolones derivatives for use in the treatment, amelioration or prevention of a viral disease

(71) Applicants: F. Hoffmann-La Roche AG, Basel (CH); Savira pharmaceuticals GmbH, Vienna (AT); European Molecular Biology Laboratory, Heidelberg (DE)

(72) Inventors: Helmut BUSCHMANN, Aachen (DE); Oliver SZOLAR, Vienna (AT); Andrea WOLKERSTORFER, Vienna (AT); Norbert HANDLER, Vienna (AT); Stephen CUSACK, Seyssinet-Pariset (FR); Robert WEIKERT, Basel (CH); Werner NEIDHART, Basel (CH)

(21) Appl. No.: 15/266,254

(22) Filed: Sep. 15, 2016

Related U.S. Application Data
(60) Provisional application No. 62/220,804, filed on Sep. 18, 2015.

(51) Int. Cl. C07D 471/04; A61K 45/06; A61K 31/437

(52) U.S. Cl. CPC C07D 471/04; A61K 31/437; A61K 45/06

(57) ABSTRACT

The present invention relates to a compound having the general formula (I), optionally in the form of a pharmaceutically acceptable salt, solvate, polymorph, prodrug, tautomer, racemate, codrug, cocrystal, enantiomer, or diastereomer or mixture thereof,

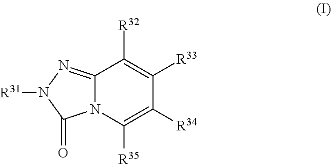

which is useful in treating, ameloriating or preventing a viral disease. Furthermore, specific combination therapies are disclosed.

### CROSS-REFERENCE TO RELATED APPLICATIONS

This application claims priority from U.S. Provisional Patent Application No. 62/220,804, filed Sep. 18, 2015, which is incorporated herein by reference in its entirety.

### FIELD OF THE INVENTION

The present invention relates to a compound having the general formula (I), optionally in the form of a pharmaceutically acceptable salt, solvate, polymorph, prodrug, tautomer, racemate, codrug, cocrystal, enantiomer, or diastereomer or mixture thereof,

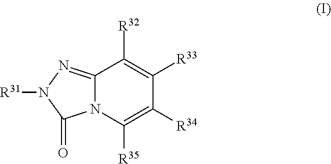

which is useful in treating, ameloriating or preventing a viral disease. Furthermore, specific combination therapies are disclosed.

### BACKGROUND OF THE INVENTION

In recent years the serious threat posed by influenza virus to worldwide public health has been highlighted by, firstly, the ongoing low level transmission to humans of the highly pathogenic avian H5N1 strain (63% mortality in infected humans, http://www.who.int/csr/disease/avian_influenza/en/) and secondly, the unexpected emergence in 2009 of a novel pandemic strain A/H1N1 that has rapidly spread around the entire world (http://www.who.int/csr/disease/swineflu/en/). Whilst the new strain is highly contagious but currently generally only gives mild illness, the future evolution of this virus is unpredictable. In a much more serious, but highly plausible scenario, H5N1 could have been more easily transmissible between humans or the new A/H1N1 could have been more virulent and could have carried the single point mutation that confers Tamiflu resistance (Neumann et al., Nature, 2009 (18; 459(7249) 931-939), as many seasonal H1N1 strains have recently done (Dharan et al., The Journal of the American Medical Association, 2009 Mar. 11; 301 (10), 1034-1041; Moscona et al., The New England Journal of Medicine, 2009 (March 5; 360(10) pp 953-956). In this case, the delay in generating and deploying a vaccine (˜6 months in the relatively favourable case of A/H1 N1 and still not a solved problem for H5N1) could have been catastrophically costly in human lives and societal disruption.

It is widely acknowledged that to bridge the period before a new vaccine becomes available and to treat severe cases, as well as to counter the problem of viral resistance, a wider choice of anti-influenza drugs is required. Development of new anti-influenza drugs has therefore again become a high priority, having been largely abandoned by the major pharmaceutical companies once the anti-neuraminidase drugs became available.

An excellent starting point for the development of antiviral medication is structural data of essential viral proteins. Thus, the crystal structure determination of e.g. the influenza virus surface antigen neuraminidase (Von Itzstein, M. et al., (1993), Nature, 363, pp. 418-423) led directly to the development of neuraminidase inhibitors with anti-viral activity preventing the release of virus from the cells, however, not the virus production. These and their derivatives have subsequently developed into the anti-influenza drugs, zanamivir (Glaxo) and oseltamivir (Roche), which are currently being stockpiled by many countries as a first line of defence against an eventual pandemic. However, these medicaments only provide a reduction in the duration of the clinical disease. Alternatively, other anti-influenza compounds such as amantadine and rimantadine target an ion channel protein, i.e., the M2 protein, in the viral membrane interfering with the uncoating of the virus inside the cell. However, they have not been extensively used due to their side effects and the rapid development of resistant virus mutants (Magden, J. et al., (2005), Appl. Microbiol. Biotechnol., 66, pp. 612-621). In addition, more unspecific viral drugs, such as ribavirin, have been shown to work for treating of influenza and other virus infections (Eriksson, B. et al., (1977), Antimicrob. Agents Chemother., 11, pp. 946-951). However, ribavirin is only approved in a few countries (Furuta et al., Antimicrobial Agents and Chemotherapy, 2005 March 49(3); 981-986), probably due to severe side effects. Clearly, new antiviral compounds are needed, preferably directed against different targets.

Influenza virus as well as Thogotovirus belong to the family of Orthomyxoviridae which, as well as the family of the Bunyaviridae, including the Hantavirus, Nairovirus, Orthobunyavirus, and Phlebovirus, are negative stranded RNA viruses. Their genome is segmented and comes in ribonucleoprotein particles that include the RNA dependent RNA polymerase which carries out (i) the initial copying of the single-stranded virion RNA (vRNA) into viral mRNAs and (ii) the vRNA replication. This enzyme, a trimeric complex composed of subunits PA, PB1 and PB2, is central to the life cycle of the virus since it is responsible for the replication and transcription of viral RNA. In previous work the atomic structure of two key domains of the polymerase, the mRNA cap-binding domain in the PB2 subunit (Guilligay et al., Antimicrobial Agents and Chemotherapy, 2005 March 49(3); pp 981-986) and the endonuclease-active site in the PA subunit (Dias et al., Nature 2009; Apr. 16; 458(7240); 914-918) have been identified and determined. These two sites are critical for the unique cap-snatching mode of transcription that is used by influenza virus to generate viral mRNAs. For the generation of viral mRNA the polymerase makes use of the so called “cap-snatching” mechanism (Plotch, S. J. et al., (1981), Cell, 23, pp. 847-858; Kukkonen, S. K. et al (2005), Arch. Virol., 150, pp. 533-556; Leahy, M. B. et al, (2005), J. Virol., 71, pp. 8347-8351; Noah, D. L. et al., (2005), Adv. Virus Res., 65, pp. 121-145). A 5′ cap (also termed an RNA cap, RNA 7-methylguanosine cap or an RNA m7G cap) is a modified guanine nucleotide that has been added to the 5′ end of each cellular messenger RNA. The 5′RNA cap consists of a terminal 7-methylguanosine residue which is linked through a 5′-5′-triphosphate bond to the first transcribed nucleotide. Upon influenza virus infection the 5′RNA cap of cellular mRNA molecules is bound by the viral polymerase complex, specifically the cap-binding domain within the PB2 subunit of the polymerase complex, and the RNA cap together with a stretch of 10 to 15 nucleotides is cleaved by the viral endonuclease which resides within the PA subunit of the viral polymerase complex. The capped RNA fragments then serve as primers for the synthesis of viral mRNA.

The cap-binding domain in the PB2 subunit of the viral polymerase has been unequivocally identified and structurally characterized by Guilligay et al., 2008. Binding the capped host cell mRNA via the cap-binding site and hence bringing the host cell mRNA strand into close spatial vicinity of the endonuclease active site is a prerequisite for the endonuclease to snatch off the cap. Therefore the cap-binding site in PB2 is essential for cap-dependent transcription by the viral RNPs and mandatory for the viral replication cycle. This together with the fact that the PB2 cap-binding domain is structually distinct from other cap binding proteins, this suggests that the ligand binding site is a good target for the development of new antiviral drugs.

Generally, the polymerase complex seems to be an appropriate antiviral drug target since it is essential for synthesis of viral mRNA and viral replication and contains several functional active sites likely to be significantly different from those found in host cell proteins (Magden, J. et al., (2005), Appl. Microbiol. Biotechnol., 66, pp. 612-621). Thus, for example, there have been attempts to interfere with the assembly of polymerase subunits by a 25-amino-acid peptide resembling the PA-binding domain within PB1 (Ghanem, A. et al., (2007), J. Virol., 81, pp. 7801-7804). Furthermore, the endonuclease activity of the polymerase has been targeted and a series of 4-substituted 2,4-dioxobutanoic acid compounds has been identified as selective inhibitors of this activity in influenza viruses (Tomassini, J. et al., (1994), Antimicrob. Agents Chemother., 38, pp. 2827-2837). In addition, flutimide, a substituted 2,6-diketopiperazine, identified in extracts of Delitschia confertaspora, a fungal species, has been shown to inhibit the endonuclease of influenza virus (Tomassini, J. et al., (1996), Antimicrob. Agents Chemother., 40, pp. 1189-1193). Moreover, there have been attempts to interfere with viral transcription by nucleoside analogs, such as 2′-deoxy-2′-fluoroguanosine (Tisdale, M. et al., (1995), Antimicrob. Agents Chemother., 39, pp. 2454-2458).

WO 2012/003392 relates to certain fused heterocyclic compounds (I) which are stated to be useful as ion channel modulators.

US 2009/0253738 discloses certain triazolopyridinone derivatives for use as stearoyl CoA desaturase inhibitors

It is an object of the present invention to identify compounds which specifically target the influenza virus cap-binding domain and hence are effective against viral diseases and which have improved pharmacological properties.

### SUMMARY OF THE INVENTION

Accordingly, in a first embodiment, the present invention provides a compound having the general formula (I) for use in the treatment, amelioration or prevention of a viral disease.

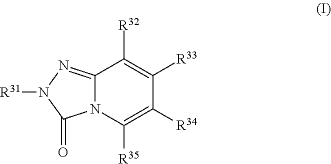

It is understood that throughout the present specification the term “a compound having the general formula (I)” encompasses pharmaceutically acceptable salts, solvates, polymorphs, prodrugs, tautomers, racemates, codrugs, cocrystals, enantiomers, or diastereomers or mixtures thereof unless mentioned otherwise.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. In the following passages different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

Definitions

The term “alkyl” refers to a saturated straight or branched or cyclic hydrocarbon group. Preferably, the term “alkyl” refers to a saturated straight or branched hydrocarbon group.

“Hal” of “halogen” represents F, Cl, Br and I.

The term “heterocyclyl” covers any ring or ring system having the indicated number of ring atoms, wherein at least one of the carbon atoms in the ring (system) has been replaced a heteroatom. If more than one heteroatom is present, they can be the same or different. The heteroatoms are preferably selected from O, N and S. The term “heterocyclyl” also covers heteroaryl rings. The term covers monocyclic rings as well as fused ring systems.

Examples of monocyclic rings include pyrrolidine; pyrrole; tetrahydrofuran; furan; thiolane; thiophene; imidazolidine; pyrazolidine; imidazole; imidazoline; pyrazole; pyrazoline; oxazolidine; isoxazolidine; oxazole; oxazoline; isoxazole; thiazolidine; isothiazolidine; thiazole; thiazoline; isothiazole; dioxolane; dithiolane; triazoles; furazan; oxadiazole; thiadiazole; dithiazole; tetrazole; piperidine; pyridine; oxane; pyran; thiane; thiopyran; piperazine; diazines (including pyrimidines); morpholine; oxazine; thiomorpholine; thiazine; dioxane; dioxine; dithiane; dithiine; triazine; trioxane; trithiane; tetrazine; azepane; azepine; oxepane; oxepine; thiepane; thiepine; homopiperazine; diazepine; and thiazepine. Fused ring systems can be envisaged as a combination of more than one of the above-mentioned monocyclic heterocyclic rings or as a combination of at least one of the above-mentioned monocyclic heterocyclic ring and a carbocyclic ring.

The term “heteroaryl” preferably refers to an aromatic ring wherein at least one of the carbon atoms in the ring (system) has been replaced a heteroatom. If more than one heteroatom is present, they can be the same or different. The heteroatoms are preferably selected from O, N and S. Examples of the heteroaryl group can be found in the list of “heterocyclyl” given above.

The term “carbocyclyl” covers any ring or ring system having the indicated number of ring atoms, which does not include heteroatoms in the ring. The term “carbocyclyl” also covers cycloalkyl and aryl rings.

The term “cycloalkyl” represents a cyclic version of “alkyl”.

The term “aryl” preferably refers to an aromatic ring. Examples include phenyl.

The term “hydrocarbon group which contains from 5 to 20 carbon atoms and optionally 1 to 4 heteroatoms selected from O, N and S and which contains at least one ring” refers to any group having 5 to 20 carbon atoms and optionally 1 to 4 heteroatoms selected from O, N and 2 as long as the group contains at least one ring. The term is also meant to include bicyclic, tricyclic and polycyclic versions thereof. If more than one ring is present, they can be separate from each other or be annelated. The ring(s) can be either carbocyclic or heterocyclic and can be saturated, unsaturated or aromatic. The carbon atoms and heteroatoms can either all be present in the one or more rings or some of the carbon atoms and/or heteroatoms can be present outside of the ring, e.g., in a linker group (such as —(CH₂)ₚ— with p=1 to 6). Examples of these groups include (optionally substituted C₅₋₉ cycloalkyl), (optionally substituted aryl) wherein the aryl group can be, for example, phenyl or naphthyl, (optionally substituted biphenyl), adamantyl, —(C₅₋₉ cycloalkyl)-aryl as well as the corresponding compounds with a linker. Furthermore, the groups given under the definitions heterocyclyl and carbocyclyl are also included.

If a compound or moiety is referred to as being “optionally substituted” it can in each instance include 1 or more of the indicated substituents, whereby the substituents can be the same or different.

The term “pharmaceutically acceptable salt” refers to a salt of a compound of the present invention. Suitable pharmaceutically acceptable salts include acid addition salts which may, for example, be formed by mixing a solution of compounds of the present invention with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compound carries an acidic moiety, suitable pharmaceutically acceptable salts thereof may include alkali metal salts (e.g., sodium or potassium salts); alkaline earth metal salts (e.g., calcium or magnesium salts); and salts formed with suitable organic ligands (e.g., ammonium, quaternary ammonium and amine cations formed using counteranions such as halide, hydroxide, carboxylate, sulfate, phosphate, nitrate, alkyl sulfonate and aryl sulfonate). Illustrative examples of pharmaceutically acceptable salts include, but are not limited to, acetate, adipate, alginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxy-ethanesulfonate, hydroxynaphthoate, iodide, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, picrate, pivalate, polygalacturonate, propionate, salicylate, stearate, sulfate, subacetate, succinate, tannate, tartrate, teoclate, tosylate, triethiodide, undecanoate, valerate, and the like (see, for example, S. M. Berge et al., “Pharmaceutical Salts”, J. Pharm. Sci., 66, pp. 1-19 (1977)).

When the compounds of the present invention are provided in crystalline form, the structure can contain solvent molecules. The solvents are typically pharmaceutically acceptable solvents and include, among others, water (hydrates) or organic solvents. Examples of possible solvates include ethanolates and iso-propanolates.

The term “codrug” refers to two or more therapeutic compounds bonded via a covalent chemical bond. A detailed definition can be found, e.g., in N. Das et al., European Journal of Pharmaceutical Sciences, 41, 2010, 571-588.

The term “cocrystal” refers to a multiple component crystal in which all components are solid under ambient conditions when in their pure form. These components co-exist as a stoichiometric or non-stoichometric ratio of a target molecule or ion (i.e., compound of the present invention) and one or more neutral molecular cocrystal formers. A detailed discussion can be found, for example, in Ning Shan et al., Drug Discovery Today, 13(9/10), 2008, 440-446 and in D. J. Good et al., Cryst. Growth Des., 9(5), 2009, 2252-2264.

The compounds of the present invention can also be provided in the form of a prodrug, namely a compound which is metabolized in vivo to the active metabolite.

### Compounds Having the General Formula (I)

The present invention provides a compound having the general formula (I):

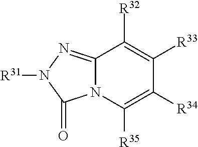

The present invention provides a compound having the general formula (I) in which the following definitions apply.

R³¹ is selected from —H and —C₁₋₆ alkyl; preferably R³¹ is —H.

R³² is selected from —H and —C₁₋₆ alkyl; preferably R³² is —H.

R³³ is selected from —H and —C₁₋₆ alkyl; preferably R³³ is —H.

R³⁴ is selected from —(X³²)ₜ (optionally substituted hydrocarbon group which contains from 5 to 20 carbon atoms and optionally 1 to 4 heteroatoms selected from O, N and S).

Preferably, R³⁴ is selected from (optionally substituted hydrocarbon group which contains from 5 to 20 carbon atoms and optionally 1 to 4 heteroatoms selected from O, N and S).

Preferably, the hydrocarbon group is a hydrocarbon group which contains from 5 to 15 carbon atoms and optionally 1 or 2 heteroatoms selected from O, N and S. More preferably, the hydrocarbon group is a hydrocarbon group which contains from 5 to 12 carbon atoms and optionally 1 or 2 heteroatoms selected from O, N and S.

In one embodiment, the hydrocarbon group which contains from 5 to 20 carbon atoms and optionally 1 to 4 heteroatoms selected from O, N and S can be selected from

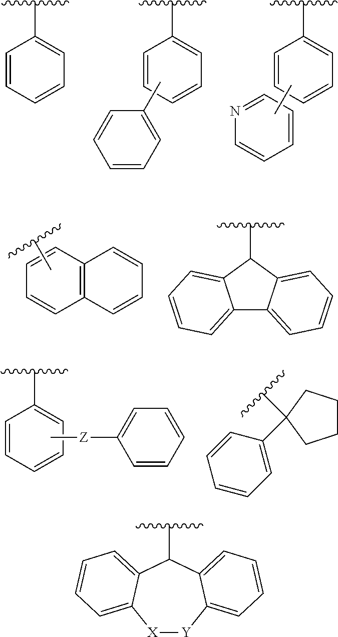

wherein

X is absent, CH₂, NH, C(O)NH, S or O;

Y is CH₂; and

Z is O or S,

wherein each of the above examples can be substituted with one or more substituents independently selected from the optional substituents which have been defined for the (optionally substituted hydrocarbon group which contains from 5 to 20 carbon atoms and optionally 1 to 4 heteroatoms selected from O, N and S).

For instance, the hydrocarbon group which contains from 5 to 20 carbon atoms and optionally 1 to 4 heteroatoms selected from O, N and S can be selected from

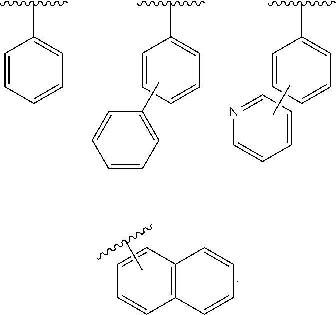

In a further embodiment, the hydrocarbon group which contains from 5 to 20 carbon atoms and optionally 1 to 4 heteroatoms selected from O, N and S can be a heterocyclyl group as defined above. Preferably, the hydrocarbon group can be a monocyclic or polycyclic heterocyclic group having 5 to 15 carbon atoms and 1 or 2 heteroatoms selected from O, N and S. More preferably, the hydrocarbon group is a heterocyclyl group which contains from 5 to 12 carbon atoms and 1 or 2 heteroatoms selected from O, N and S. Examples of the heterocyclyl group which can be employed as the hydrocarbon group include those given in the definition section above. Preferred examples include pyrimidine, 3-cyanopyridines and 1,3,5-triazines, pyrimidine is particularly preferred.

The above shown moieties can be optionally substituted in any available position by one or more substituents which are given for the hydrocarbon group. In one embodiment, the above shown moieties can be optionally substituted in any available position by one or more substituents which are independently selected from —C₁₋₆ alkyl, -halogen, —O—C₁₋₆ alkyl, —CF₃, —COF₃, —CN, and —OH.

R³⁵ is selected from —H and —C₁₋₆ alkyl; preferably R³⁵ is —H.

R³⁶ is selected from —H, (optionally substituted C₁₋₆ alkyl), (optionally substituted C₃₋₉ carbocyclyl), —C₁₋₄ alkyl (optionally substituted C₃₋₉ carbocyclyl), (optionally substituted heterocyclyl having 3 to 9 ring atoms), and —C₁₋₄ alkyl-(optionally substituted heterocyclyl having 3 to 9 ring atoms); preferably R³⁶ is selected from —H, and —(C₁₋₆ alkyl); more preferably R³⁶ is —H.

R³⁸ is selected from —H, (optionally substituted C₁₋₆ alkyl), (optionally substituted C₃₋₉ carbocyclyl), —C₁₋₄ alkyl (optionally substituted C₃₋₉ carbocyclyl), (optionally substituted heterocyclyl having 3 to 9 ring atoms), and —C₁₋₄ alkyl (optionally substituted heterocyclyl having 3 to 9 ring atoms); preferably R³⁸ is selected from —H, (optionally substituted C₁₋₆ alkyl), (optionally substituted C₃₋₇ carbocyclyl), and (optionally substituted heterocyclyl having 3 to 7 ring atoms), more preferably R³⁸ is selected from —H, (optionally substituted C₁₋₆ alkyl), (optionally substituted C₅₋₆ carbocyclyl), and (optionally substituted heterocyclyl having 5 to 6 ring atoms).

X³² is selected from NR³⁶, N(R³⁶)C(O), C(O)NR³⁶, O, C(O), C(O)O, OC(O), N(R³⁶)SO₂, SO₂N(R³⁶), S, SO, and SO₂; preferably X³² is selected from N(R³⁶)C(O), C(O)NR³⁶, O, C(O), C(O)O, and OC(O); more preferably X³² is selected from N(R³⁶)C(O), C(O)NR³⁶, O, C(O)O, and OC(O).

s is 0 to 4; preferably s is 0.

t is 0 or 1; preferably t is 0.

The alkyl group can be optionally substituted with one or more substituents which are independently selected from -halogen, —CN, —CF₃, —COF₃, —(CH₂)ₛ—X³²—R³⁸, —C₃₋₉ carbocyclyl, and (heterocyclyl having 3 to 9 ring atoms). The alkyl group in R³⁶ and R³⁸ is preferably unsubstituted.

The hydrocarbon group can be optionally substituted with one or more substituents which are independently selected from -halogen, —CN, —CF₃, —COF₃, —(CH₂)ₛ—X³²—R³⁸, —C₁₋₆ alkyl, —C₃₋₉ carbocyclyl which is optionally substituted by —OH or -Hal, —C₁₋₄ alkyl-C₃₋₉ carbocyclyl which is optionally substituted by —OH or -Hal, (heterocyclyl having 3 to 9 ring atoms which is optionally substituted by —OH or -Hal), and —C₁₋₄ alkyl-(heterocyclyl having 3 to 9 ring atoms which is optionally substituted by —OH or -Hal). Preferred examples of the substituents include —C₁₋₆ alkyl, halogen, —O—C₁₋₆ alkyl, —CF₃, —COF₃, —CN, and —OH.

The heterocyclyl group can be optionally substituted with one or more substituents which are independently selected from -halogen, —CN, —CF₃, —COF₃, —(CH₂)ₛ—X³²—R³⁸, —C₁₋₆ alkyl, —C₃₋₉ carbocyclyl which is optionally substituted by —OH or -Hal, —C₁₋₄ alkyl-C₃₋₉ carbocyclyl which is optionally substituted by —OH or -Hal, (heterocyclyl having 3 to 9 ring atoms which is optionally substituted by —OH or -Hal), and —C₁₋₄ alkyl-(heterocyclyl having 3 to 9 ring atoms which is optionally substituted by —OH or -Hal). The heterocyclyl group in R³⁶ and R³⁸ is preferably unsubstituted.

The carbocyclyl group can be optionally substituted with one or more substituents which are independently selected from -halogen, —CN, —CF₃, —COF₃, —(CH₂)ₛ-—X³²—R³⁸, —C₁₋₆ alkyl, —C₃₋₉ carbocyclyl which is optionally substituted by —OH or -Hal, —C₁₋₄ alkyl-C₃₋₉ carbocyclyl which is optionally substituted by —OH or -Hal, (heterocyclyl having 3 to 9 ring atoms which is optionally substituted by —OH or -Hal), and —C₁₋₄ alkyl-(heterocyclyl having 3 to 9 ring atoms which is optionally substituted by —OH or -Hal). The carbocyclyl group in R³⁶ and R³⁸ is preferably unsubstituted.

The compounds of the present invention can be administered to a patient in the form of a pharmaceutical composition which can optionally comprise one or more pharmaceutically acceptable excipient(s) and/or carrier(s).

The compounds of the present invention can be administered by various well known routes, including oral, rectal, intragastrical, intracranial and parenteral administration, e.g. intravenous, intramuscular, intranasal, intradermal, subcutaneous, and similar administration routes. Oral, intranasal and parenteral administration are particularly preferred. Depending on the route of administration different pharmaceutical formulations are required and some of those may require that protective coatings are applied to the drug formulation to prevent degradation of a compound of the invention in, for example, the digestive tract.

Thus, preferably, a compound of the invention is formulated as a syrup, an infusion or injection solution, a spray, a tablet, a capsule, a capslet, lozenge, a liposome, a suppository, a plaster, a band-aid, a retard capsule, a powder, or a slow release formulation. Preferably the diluent is water, a buffer, a buffered salt solution or a salt solution and the carrier preferably is selected from the group consisting of cocoa butter and vitebesole.

Particular preferred pharmaceutical forms for the administration of a compound of the invention are forms suitable for injectionable use and include sterile aqueous solutions or dispersions and sterile powders for the extemporaneous preparation of sterile injectable solutions or dispersion. In all cases the final solution or dispersion form must be sterile and fluid. Typically, such a solution or dispersion will include a solvent or dispersion medium, containing, for example, water-buffered aqueous solutions, e.g. biocompatible buffers, ethanol, polyol, such as glycerol, propylene glycol, polyethylene glycol, suitable mixtures thereof, surfactants or vegetable oils. A compound of the invention can also be formulated into liposomes, in particular for parenteral administration. Liposomes provide the advantage of increased half life in the circulation, if compared to the free drug and a prolonged more even release of the enclosed drug.

Sterilization of infusion or injection solutions can be accomplished by any number of art recognized techniques including but not limited to addition of preservatives like anti-bacterial or anti-fungal agents, e.g. parabene, chlorobutanol, phenol, sorbic acid or thimersal. Further, isotonic agents, such as sugars or salts, in particular sodium chloride may be incorporated in infusion or injection solutions.

Production of sterile injectable solutions containing one or several of the compounds of the invention is accomplished by incorporating the respective compound in the required amount in the appropriate solvent with various ingredients enumerated above as required followed by sterilization. To obtain a sterile powder the above solutions are vacuum-dried or freeze-dried as necessary. Preferred diluents of the present invention are water, physiological acceptable buffers, physiological acceptable buffer salt solutions or salt solutions. Preferred carriers are cocoa butter and vitebesole. Excipients which can be used with the various pharmaceutical forms of a compound of the invention can be chosen from the following non-limiting list:

a) binders such as lactose, mannitol, crystalline sorbitol, dibasic phosphates, calcium phosphates, sugars, microcrystalline cellulose, carboxymethyl cellulose, hydroxyethyl cellulose, polyvinyl pyrrolidone and the like;

b) lubricants such as magnesium stearate, talc, calcium stearate, zinc stearate, stearic acid, hydrogenated vegetable oil, leucine, glycerids and sodium stearyl fumarates,

c) disintegrants such as starches, croscaramellose, sodium methyl cellulose, agar, bentonite, alginic acid, carboxymethyl cellulose, polyvinyl pyrrolidone and the like.

In one embodiment the formulation is for oral administration and the formulation comprises one or more or all of the following ingredients: pregelatinized starch, talc, povidone K 30, croscarmellose sodium, sodium stearyl fumarate, gelatin, titanium dioxide, sorbitol, monosodium citrate, xanthan gum, titanium dioxide, flavoring, sodium benzoate and saccharin sodium.

If a compound of the invention is administered intranasally in a preferred embodiment, it may be administered in the form of a dry powder inhaler or an aerosol spray from a pressurized container, pump, spray or nebulizer with the use of a suitable propellant, e.g., dichlorodifluoromethane, trichlorofluoromethane, dichlorotetrafluoroethane, a hydrofluoro-alkane such as 1,1,1,2-tetrafluoroethane (HFA 134A™) or 1,1,1,2,3,3,3-heptafluoropropane (HFA 227EA™), carbon dioxide, or another suitable gas. The pressurized container, pump, spray or nebulizer may contain a solution or suspension of the compound of the invention, e.g., using a mixture of ethanol and the propellant as the solvent, which may additionally contain a lubricant, e.g., sorbitan trioleate.

Other suitable excipients can be found in the Handbook of Pharmaceutical Excipients, published by the American Pharmaceutical Association, which is herein incorporated by reference.

It is to be understood that depending on the severity of the disorder and the particular type which is treatable with one of the compounds of the invention, as well as on the respective patient to be treated, e.g. the general health status of the patient, etc., different doses of the respective compound are required to elicit a therapeutic or prophylactic effect. The determination of the appropriate dose lies within the discretion of the attending physician. It is contemplated that the dosage of a compound of the invention in the therapeutic or prophylactic use of the invention should be in the range of about 0.1 mg to about 1 g of the active ingredient (i.e. compound of the invention) per kg body weight. However, in a preferred use of the present invention a compound of the invention is administered to a subject in need thereof in an amount ranging from 1.0 to 500 mg/kg body weight, preferably ranging from 1 to 200 mg/kg body weight. The duration of therapy with a compound of the invention will vary, depending on the severity of the disease being treated and the condition and idiosyncratic response of each individual patient. In one preferred embodiment of a prophylactic or therapeutic use, between 100 mg to 200 mg of the compound is orally administered to an adult per day, depending on the severity of the disease and/or the degree of exposure to disease carriers.

As is known in the art, the pharmaceutically effective amount of a given composition will also depend on the administration route. In general the required amount will be higher, if the administration is through the gastrointestinal tract, e.g., by suppository, rectal, or by an intragastric probe, and lower if the route of administration is parenteral, e.g., intravenous. Typically, a compound of the invention will be administered in ranges of 50 mg to 1 g/kg body weight, preferably 100 mg to 500 mg/kg body weight, if rectal or intragastric administration is used and in ranges of 10 to 100 mg/kg body weight, if parenteral administration is used.

If a person is known to be at risk of developing a disease treatable with a compound of the invention, prophylactic administration of the biologically active blood serum or the pharmaceutical composition according to the invention may be possible. In these cases the respective compound of the invention is preferably administered in above outlined preferred and particular preferred doses on a daily basis. Preferably, from 0.1 mg to 1 g/kg body weight once a day, preferably 10 to 200 mg/kg body weight. This administration can be continued until the risk of developing the respective viral disorder has lessened. In most instances, however, a compound of the invention will be administered once a disease/disorder has been diagnosed. In these cases it is preferred that a first dose of a compound of the invention is administered one, two, three or four times daily.

The compounds of the present invention are particularly useful for treating, ameliorating, or preventing viral diseases. The type of viral disease is not particularly limited. Examples of possible viral diseases include, but are not limited to, viral diseases which are caused by Poxviridae, Herpesviridae, Adenoviridae, Papillomaviridae, Polyomaviridae, Parvoviridae, Hepadnaviridae, Retroviridae, Reoviridae, Filoviridae, Paramyxoviridae, Rhabdoviridae, Orthomyxoviridae, Bunyaviridae, Arenaviridae, Coronaviridae, Picornaviridae, Hepeviridae, Caliciviridae, Astroviridae, Togaviridae, Flaviviridae, Deltavirus, Bornaviridae, and prions. Preferably viral diseases which are caused by Herpesviridae, Retroviridae, Filoviridae, Paramyxoviridae, Rhabdoviridae, Orthomyxoviridae, Bunyaviridae, Arenaviridae, Coronaviridae, Picornaviridae, Togaviridae, Flaviviridae, more preferably viral diseases which are caused by orthomyxoviridae.

Examples of the various viruses are given in the following table.

| Family | Virus (preferred examples) |
|---|---|
| Poxviridae | Smallpox virus |
| | Molluscum contagiosum virus |
| Herpesviridae | Herpes simplex virus |
| | Varicella zoster virus |
| | Cytomegalovirus |
| | Epstein Barr virus |
| | Kaposi's sarcoma-associated |
| | herpesvirus |
| Adenoviridae | Human adenovirus A-F |
| Papillomaviridae | Papillomavirus |
| Polyomaviridae | BK-virus |
| | JC-Virsu |
| Parvoviridae | B19 virus |
| | Adeno associated virus 2/3/5 |
| Hepadnaviridae | Hepatitis B virus |
| Retroviridae | Human immunodeficiency virus |
| | types 1/2 |
| | Human T-cell leukemia virus |
| | Human foamy virus |
| Reoviridae | Reovirus 1/2/3 |
| | Rotavirus A/B/C |
| | Colorado tick fever virus |
| Filoviridae | Ebola virus |
| | Marburg virus |
| Paramyxoviridae | Parainfluenza virus 1-4 |
| | Mumps virus |
| | Measles virus |
| | Respiratory syncytial virus |
| | Hendravirus |
| Rhabdoviridae | Vesicular stomatitis virus |
| | Rabies virus |
| | Mokola virus |
| | European bat virus |
| | Duvenhage virus |
| Orthomyxoviridae | Influenza virus types A-C |
| Bunyaviridae | California encephalitis virus |
| | La Crosse virus |
| | Hantaan virus |
| | Puumala virus |
| | Sin Nombre virus |
| | Seoul virus |
| | Crimean-Congo hemorrhagic fever virus |
| | Sakhalin virus |
| | Rift valley virus |
| | Sandfly fever virus |
| | Uukuniemi virus |
| Arenaviridae | Lassa virus |
| | Lymphocytic choriomeningitis virus |
| | Guanarito virus |
| | Junin virus, |
| | Machupo virus |
| | Sabia virus |
| Coronaviridae | Human coronavirus |
| Picornaviridae | Human enterovirus types A-D (Poliovirus, |
| | Echovirus, Coxsackie virus A/B) |
| | Rhinovirus types A/B/C |
| | Hepatitis A virus |
| | Parechovirus |
| | Food and mouth disease virus |
| Hepeviridae | Hepatitis E virus |
| Caliciviridae | Norwalk virus |
| | Sapporo virus |
| Astroviridae | Human astrovirus 1 |
| Togaviridae | Ross River virus |
| | Chikungunya virus |
| | O'nyong-nyong virus |
| | Rubella virus |
| Flaviviridae | Tick-borne encephalitis virus |
| | Dengue virus |
| | Yellow Fever virus |
| | Japanese encephalitis virus |
| | Murray Valley virus |
| | St. Louis encephalitis virus |
| | West Nile virus |
| | Hepatitis C virus |
| | Hepatitis G virus |
| | Hepatitis GB virus |
| Deltavirus | Hepatitis deltavirus |
| Bornaviridae | Bornavirus |
| Prions | |

Preferably the compounds of the present invention are employed to treat influenza. Within the present invention, the term “influenza” includes influenza A, B, C, isavirus and thogotovirus and also covers bird flu and swine flu. The subject to be treated is not particularly restricted and can be any vertebrate, such as birds and mammals (including humans).

Without wishing to be bound by theory it is assumed that the compounds of the present invention are capable of inhibiting binding of host mRNA cap structures to the cap-binding domain (CBD), particularly of the influenza virus. More specifically it is assumed that they directly interfere with the CBD of the influenza PB2 protein. However, delivery of a compound into a cell may represent a problem depending on, e.g., the solubility of the compound or its capabilities to cross the cell membrane. The present invention not only shows that the claimed compounds have in vitro polymerase inhibitory activity but also cellular antiviral activity.

A possible measure of the cellular antiviral activity of the compounds having the formula (I) is the CPE assay disclosed herein. Preferably the compounds exhibit a % reduction of at least about 30% at 50 μM. In this connection, the reduction in the virus-mediated cytopathic effect (CPE) upon treatment with the compounds was calculated as follows: The cell viability of infected-treated and uninfected-treated cells was determined using an ATP-based cell viability assay (Promega). The response in relative luminescent units (RLU) of infected-untreated samples was subtracted from the response (RLU) of the infected-treated samples and then normalized to the viability of the corresponding uninfected sample resulting in % CPE reduction. Preferably the compounds exhibit an IC₅₀ of at least about 45 μM, more preferably at least about 10 μM, in the CPE assay. The half maximal inhibitory concentration (IC₅₀) is a measure of the effectiveness of a compound in inhibiting biological or biochemical function and was calculated from the RLU response in a given concentration series ranging from maximum 100 μM to at least 100 nM.

The activity of the compounds having the formula (I) can also be measured by the Cap Fluorescence-Polarization Ligand Displacement (CapFP-LD) assay as described herein.

The compounds having the general formula (I) can be used in combination with one or more other medicaments. The type of the other medicaments is not particularly limited and will depend on the disorder to be treated. Preferably the other medicament will be a further medicament which is useful in treating, ameloriating or preventing a viral disease, more preferably a further medicament which is useful in treating, ameloriating or preventing influenza.

The following combinations of medicaments are envisaged as being particularly suitable:
(i) The combination of endonuclease and cap binding inhibitors (particularly targeting influenza). The endonuclease inhibitors are not particularly limited and can be any endonuclease inhibitor, particularly any viral endonuclease inhibitor.
   Widespread resistance to both classes of licensed influenza antivirals (M2 ion channel inhibitors (adamantanes) and neuraminidase inhibitors (Oseltamivir)) occurs in both pandemic and seasonal viruses, rendering these drugs to be of marginal utility in the treatment modality. For M2 ion channel inhibitors, the frequency of viral resistance has been increasing since 2003 and for seasonal influenza A/H3N2, adamantanes are now regarded as ineffective. Virtually all 2009 H1N1 and seasonal H3N2 strains are resistant to the adamantanes (rimantadine and amantadine), and the majority of seasonal H1N1 strains are resistant to oseltamivir, the most widely prescribed neuraminidase inhibitor (NAI). For oseltamivir the WHO reported on significant emergence of influenza A/H1N1 resistance starting in the influenza season 2007/2008; and for the second and third quarters of 2008 in the southern hemisphere. Even more serious numbers were published for the fourth quarter of 2008 (northern hemisphere) where 95% of all tested isolates revealed no Oseltamivir-susceptibility. Considering the fact that now most national governments have been stockpiling Oseltamivir as part of their influenza pandemic preparedness plan, it is obvious that the demand for new, effective drugs is growing significantly. To address the need for more effective therapy, preliminary studies using double or even triple combinations of antiviral drugs with different mechanisms of action have been undertaken. Adamantanes and neuraminidase inhibitors in combination were analysed in vitro and in vivo and found to act highly synergistically. However, it is known that for both types of antivirals resistant viruses emerge rather rapidly and this issue is not tackled by combining these established antiviral drugs.
   Influenza virus polymerase inhibitors are novel drugs targeting the transcription activity of the polymerase. Selective inhibitors against the cap-binding and endonuclease active sites of the viral polymerase severely attenuate virus infection by stopping the viral reproductive cycle. These two targets are located within distinct subunits of the polymerase complex and thus represent unique drug targets. Due to the fact that both functions are required for the so-called “cap-snatching” mechanism mandatory for viral transcription, concurrent inhibition of both functions is expected to act highly synergistically. This highly efficient drug combination would result in lower substance concentrations and hence improved dose-response-relationships and better side effect profiles.
   Both of these active sites are composed of identical residues in all influenza A strains (e.g., avian and human) and hence this high degree of sequence conservation underpins the perception that these targets are not likely to trigger rapid resistant virus generation. Thus, endonuclease and cap-binding inhibitors individually and in combination are ideal drug candidates to combat both seasonal and pandemic influenza, irrespectively of the virus strain.
   The combination of an endonuclease inhibitor and a cap-binding inhibitor or a dual specific polymerase inhibitor targeting both the endonuclease active site and the cap-binding domain would be effective against virus strains resistant against adamantanes and neuraminidase inhibitors and moreover combine the advantage of low susceptibility to resistance generation with activity against a broad range of virus strains.
(ii) The combination of inhibitors of different antiviral targets (particularly targeting influenza) focusing on the combination with (preferably influenza) polymerase inhibitors as dual or multiple combination therapy. Influenza virus polymerase inhibitors are novel drugs targeting the transcription activity of the polymerase. Selective inhibitors against the cap-binding and endonuclease active sites of the viral polymerase severely attenuate virus infection by stopping the viral reproductive cycle. The combination of a polymerase inhibitor specifically addressing a viral intracellular target with an inhibitor of a different antiviral target is expected to act highly synergistically. This is based on the fact that these different types of antiviral drugs exhibit completely different mechanisms of action and pharmacokinetics properties which act advantageously and synergistically on the antiviral efficacy of the combination.
   This highly efficient drug combination would result in lower substance concentrations and hence improved dose-response-relationships and better side effect profiles. Moreover, advantages described under (i) for polymerase inhibitors would prevail for combinations of inhibitors of different antiviral targets with polymerase inhibitors.
   Typically at least one compound selected from the first group of polymerase inhibitors is combined with at least one compound selected from the second group of polymerase inhibitors.
   The first group of polymerase inhibitors which can be used in this type of combination therapy includes, but is not limited to, the compounds having the general formula (I) described below, the compounds having the general formula ((I)) described above and/or the compounds disclosed in WO2011/000566.
   The second group of polymerase inhibitors which can be used in this type of combination therapy includes, but is not limited to, compounds disclosed in WO 2010/110231, WO 2010/110409, WO 2006/030807 and U.S. Pat. No. 5,475,109 as well as flutimide and analogues, favipiravir and analogues, epigallocatechin gallate and analogues, as well as nucleoside analogs such as ribavirine.
(iii) The combination of polymerase inhibitors with neuramidase inhibitors
   Influenza virus polymerase inhibitors are novel drugs targeting the transcription activity of the polymerase. Selective inhibitors against the cap-binding and endonuclease active sites of the viral polymerase severely attenuate virus infection by stopping the viral reproductive cycle. The combination of a polymerase inhibitor specifically addressing a viral intracellular target with an inhibitor of a different extracellular antiviral target, especially the (e.g., viral) neuraminidase is expected to act highly synergistically. This is based on the fact that these different types of antiviral drugs exhibit completely different mechanisms of action and pharmacokinetic properties which act advantageously and synergistically on the antiviral efficacy of the combination.
   This highly efficient drug combination would result in lower substance concentrations and hence improved dose-response-relationships and better side effect profiles. Moreover, advantages described under (i) for polymerase inhibitors would prevail for combinations of inhibitors of different antiviral targets with polymerase inhibitors.
   Typically at least one compound selected from the above mentioned first group of polymerase inhibitors is combined with at least one neuramidase inhibitor.
   The neuraminidase inhibitor (particularly influenza neuramidase inhibitor) is not specifically limited. Examples include zanamivir, oseltamivir, peramivir, KDN DANA, FANA, and cyclopentane derivatives.
(iv) The combination of polymerase inhibitors with M2 channel inhibitors
   Influenza virus polymerase inhibitors are novel drugs targeting the transcription activity of the polymerase. Selective inhibitors against the cap-binding and endonuclease active sites of the viral polymerase severely attenuate virus infection by stopping the viral reproductive cycle. The combination of a polymerase inhibitor specifically addressing a viral intracellular target with an inhibitor of a different extracellular and cytoplasmic antiviral target, especially the viral M2 ion channel, is expected to act highly synergistically. This is based on the fact that these different types of antiviral drugs exhibit completely different mechanisms of action and pharmacokinetic properties which act advantageously and synergistically on the antiviral efficacy of the combination.
   This highly efficient drug combination would result in lower substance concentrations and hence improved dose-response-relationships and better side effect profiles. Moreover, advantages described under (i) for polymerase inhibitors would prevail for combinations of inhibitors of different antiviral targets with polymerase inhibitors. Typically at least one compound selected from the above mentioned first group of polymerase inhibitors is combined with at least one M2 channel inhibitor.
   The M2 channel inhibitor (particularly influenza M2 channel inhibitor) is not specifically limited. Examples include amantadine and rimantadine.
(v) The combination of polymerase inhibitors with alpha glucosidase inhibitors
   Influenza virus polymerase inhibitors are novel drugs targeting the transcription activity of the polymerase. Selective inhibitors against the cap-binding and endonuclease active sites of the viral polymerase severely attenuate virus infection by stopping the viral reproductive cycle. The combination of a polymerase inhibitor specifically addressing a viral intracellular target, with an inhibitor of a different extracellular target, especially alpha glucosidase, is expected to act highly synergistically. This is based on the fact that these different types of antiviral drugs exhibit completely different mechanisms of action and pharmacokinetic properties which act advantageously and synergistically on the antiviral efficacy of the combination.
   This highly efficient drug combination would result in lower substance concentrations and hence improved dose-response-relationships and better side effect profiles. Moreover, advantages described under (i) for polymerase inhibitors would prevail for combinations of inhibitors of different antiviral targets with polymerase inhibitors.
   Typically at least one compound selected from the above mentioned first group of polymerase inhibitors is combined with at least one alpha glucosidase inhibitor.
   The alpha glucosidase inhibitor (particularly influenza alpha glucosidase inhibitor) is not specifically limited. Examples include the compounds described in Chang et al., Antiviral Research 2011, 89, 26-34.
(vi) The combination of polymerase inhibitors with ligands of other influenza targets
   Influenza virus polymerase inhibitors are novel drugs targeting the transcription activity of the polymerase. Selective inhibitors against the cap-binding and endonuclease active sites of the viral polymerase severely attenuate virus infection by stopping the viral reproductive cycle. The combination of a polymerase inhibitor specifically addressing a viral intracellular target with an inhibitor of different extracellular, cytoplasmic or nucleic antiviral targets is expected to act highly synergistically. This is based on the fact that these different types of antiviral drugs exhibit completely different mechanisms of action and pharmacokinetic properties which act advantageously and synergistically on the antiviral efficacy of the combination.
   This highly efficient drug combination would result in lower substance concentrations and hence improved dose-response-relationships and better side effect profiles. Moreover, advantages described under (i) for polymerase inhibitors would prevail for combinations of inhibitors of different antiviral targets with polymerase inhibitors.
   Typically at least one compound selected from the above mentioned first group of polymerase inhibitors is combined with at least one ligand of another influenza target.
   The ligand of another influenza target is not specifically limited. Examples include compounds acting on the sialidase fusion protein, e.g. Fludase (DAS181), siRNAs and phosphorothioate oligonucleotides, signal transduction inhibitors (ErbB tyrosine kinase, Abl kinase family, MAP kinases, PKCa-mediated activation of ERK signaling as well as interferon (inducers).
(vii) The combination of (preferably influenza) polymerase inhibitors with a compound used as an adjuvance to minimize the symptoms of the disease (antibiotics, anti-inflammatory agents like COX inhibitors (e.g., COX-1/COX-2 inhibitors, selective COX-2 inhibitors), lipoxygenase inhibitors, EP ligands (particularly EP4 ligands), bradykinin ligands, and/or cannabinoid ligands (e.g., CB2 agonists). Influenza virus polymerase inhibitors are novel drugs targeting the transcription activity of the polymerase. Selective inhibitors against the cap-binding and endonuclease active sites of the viral polymerase severely attenuate virus infection by stopping the viral reproductive cycle. The combination of a polymerase inhibitor specifically addressing a viral intracellular target with an compound used as an adjuvance to minimize the symptoms of the disease address the causative and symptomatic pathological consequences of viral infection. This combination is expected to act synergistically because these different types of drugs exhibit completely different mechanisms of action and pharmacokinetic properties which act advantageously and synergistically on the antiviral efficacy of the combination.
   This highly efficient drug combination would result in lower substance concentrations and hence improved dose-response-relationships and better side effect profiles. Moreover, advantages described under (i) for polymerase inhibitors would prevail for combinations of inhibitors of different antiviral targets with polymerase inhibitors.

The present invention not only shows that the compounds have in vitro polymerase inhibitory activity but also in vivo antiviral activity.

Various modifications and variations of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

The following examples are merely illustrative of the present invention and should not be construed to limit the scope of the invention as indicated by the appended claims in any way.

### EXAMPLES

Cytopathic Effect (CPE) Assay

The influenza A virus (IAV) was obtained from American Tissue Culture Collection (A/Aichi/2/68 (H3N2); VR-547). Virus stocks were prepared by propagation of virus on Mardin-Darby canine kidney (MDCK; ATCC CCL-34) cells and infectious titres of virus stocks were determined by the 50% tissue culture infective dose (TCID₅₀) analysis as described in Reed, L. J., and H. Muench., Am. J. Hyg. 1938, 27, 493-497.

MDCK cells were seeded in 96-well plates at 2×10⁴ cells/well using DMEM/Ham's F-12 (1:1) medium containing 10% foetal bovine serum (FBS), 2 mM L-glutamine and 1% antibiotics (all from PAA). Until infection, the cells were incubated for 5 hrs at 37° C., 5.0% CO₂ to form a ˜80% confluent monolayer on the bottom of the well. Each test compound was dissolved in DMSO and generally tested at 25 μM and 250 μM. In those cases where the compounds were not soluble at that concentration they were tested at the highest soluble concentration. The compounds were diluted in infection medium (DMEM/Ham's F-12 (1:1) containing 5 μg/ml trypsin, and 1% antibiotics) for a final plate well DMSO concentration of 1%. The virus stock was diluted in infection medium (DMEM/Ham's F-12 (1:1) containing 5 pg/ml Trypsin, 1% DMSO, and 1% antibiotics) to a theoretical multiplicity of infection (MOI) of 0.05.

After removal of the culture medium and one washing step with PBS, virus and compound were added together to the cells. In the wells used for cytotoxicity determination (i.e. in the absence of viral infection), no virus suspension was added. Instead, infection medium was added. Each treatment was conducted in two replicates. After incubation at 37° C., 5% CO₂ for 48 hrs, each well was observed microscopically for apparent cytotoxicity, precipitate formation, or other notable abnormalities. Then, cell viability was determined using CellTiter-Glo luminescent cell viability assay (Promega). The supernatant was removed carefully and 65 μl of the reconstituted reagent were added to each well and incubated with gentle shaking for 15 min at room temperature. Then, 60 μl of the solution was transferred to an opaque plate and luminescence (RLU) was measured using Synergy HT plate reader (Biotek).

Relative cell viability values of uninfected-treated versus uninfected-untreated cells were used to evaluate cytotoxicity of the compounds. Substances with a relative viability below 80% at the tested concentration were regarded as cytotoxic and retested at lower concentrations.

Reduction in the virus-mediated cytopathic effect (CPE) upon treatment with the compounds was calculated as follows: The response (RLU) of infected-untreated samples was subtracted from the response (RLU) of the infected-treated samples and then normalized to the viability of the corresponding uninfected sample resulting in % CPE reduction. The half maximal inhibitory concentration (IC₅₀) is a measure of the effectiveness of a compound in inhibiting biological or biochemical function and was calculated from the RLU response in a given concentration series ranging from maximum 100 μM to at least 100 nM.

Cap Fluorescence-Polarization Ligand Displacement (CapFP-LD) Assay

The expression construct for PB2 cap binding domain (PB2-CBD) (residues 318-483) of the avian influenza strain A/duck/Shantou/4610/2003(H5N1) was synthesized by Geneart AG. Purified protein was kindly provided by Stephen Cusack and his co-workers (EMBL Grenoble; Guilligay et al., 2008). The PB2-CBD concentration was determined by OD₂₈₀ measurement using the extinction coefficient of 6990 M^{-1.}cm⁻¹ at 280 nm, m⁷GTP-5FAM (Jena Bioscience) was used as a fluorescent tracer. The concentrations of tracer and receptor were chosen according to their K_{d} value of 0.42 μM determined in assay buffer (10 mM HEPES pH 7.4, 100 mM NaAc, 10 mM Mg(Ac)₂, 0.005% (v/v) protein-grade TWEEN 20) (Nikolovska-Coleska et al., 2004). A series of 2-fold dilutions of compound were prepared, transferred to 384-well plates (Corning #3676) at a final DMSO concentration of 10% (v/v). The tracer/protein mixture was added to a final concentration of 2 μM and 1.2 μM respectively. The plates were sealed, incubated and then shaken for 30 min before FP was measured. The data was analyzed using GraphPad Prism to determine IC₅₀ values and 95% confidence intervals using a 4-parameter logistic equation. Positive and negative controls were included to define top and bottom for curve fitting.

In the following, the compounds were prepared according to the general schemes, unless a specific synthesis method is given.

| Formula, no. | Fₚ | CPE |
|---|---|---|
| 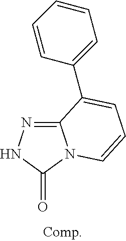 [SMILES: O=c1[nH]nc2c(-c3ccccc3)cccn12] | >50 μmol | >50 μmol |
| 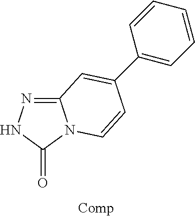 [SMILES: O=c1[nH]nc2cc(-c3ccccc3)ccn12] | >50 μmol | >50 μmol |
| 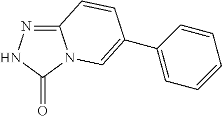 [SMILES: O=c1[nH]nc2ccc(-c3ccccc3)cn12] | Kᵢ = 33.2 μM | >50 μmol |
| 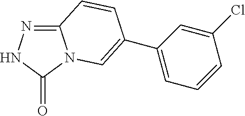 [SMILES: O=c1[nH]nc2ccc(-c3cccc(Cl)c3)cn12] | Kᵢ = 19.3 μM | >50 μmol |
| 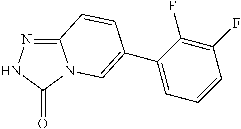 [SMILES: O=c1[nH]nc2ccc(-c3cccc(F)c3F)cn12] | Kᵢ = 26.9 μM | >50 μmol |
| 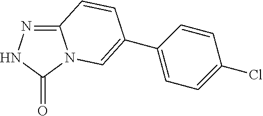 [SMILES: O=c1[nH]nc2ccc(-c3ccc(Cl)cc3)cn12] | Kᵢ = 2.8 μM | >50 μmol |
| 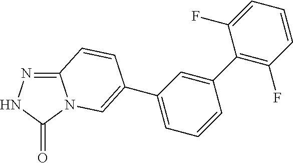 [SMILES: O=c1[nH]nc2ccc(-c3cccc(-c4c(F)cccc4F)c3)cn12] | Kᵢ = 13.7 μM | IC₅₀ = 34.9 μM |
| 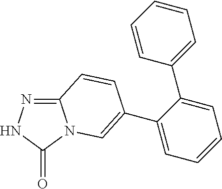 [SMILES: O=c1[nH]nc2ccc(-c3ccccc3-c3ccccc3)cn12] | >50 μmol | >50 μmol |
| 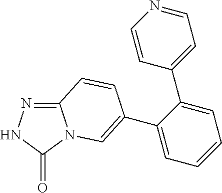 [SMILES: O=c1[nH]nc2ccc(-c3ccccc3-c3ccncc3)cn12] | >50 μmol | >50 μmol |
| 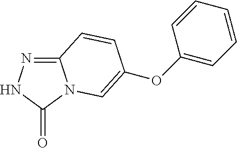 [SMILES: O=c1[nH]nc2ccc(Oc3ccccc3)cn12] | Kᵢ = 415.6 μM | >50 μmol |
| 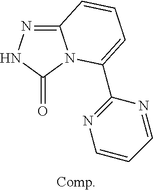 [SMILES: O=c1[nH]nc2cccc(-c3ncccn3)n12] | >50 μmol | >50 μmol |
| 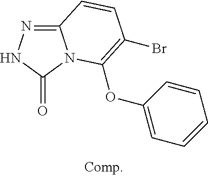 [SMILES: O=c1[nH]nc2ccc(Br)c(Oc3ccccc3)n12] | >50 μmol | >50 μmol |
| 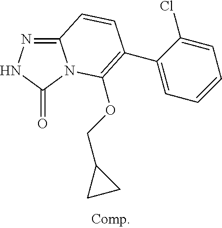 [SMILES: O=c1[nH]nc2ccc(-c3ccccc3Cl)c(OCC3CC3)n12] | >50 μmol | >50 μmol |
| 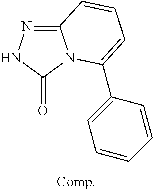 [SMILES: O=c1[nH]nc2cccc(-c3ccccc3)n12] | >50 μmol | >50 μmol |
| 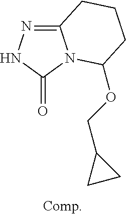 [SMILES: O=c1[nH]nc2n1C(OCC1CC1)CCC2] | >50 μmol | >50 μmol |
| 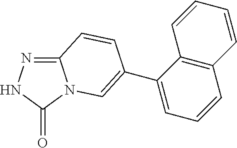 [SMILES: O=c1[nH]nc2ccc(-c3cccc4ccccc34)cn12] | Kᵢ = 13.3 μM | >50 μmol |
| 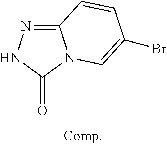 [SMILES: O=c1[nH]nc2ccc(Br)cn12] | Kᵢ = 313.7 μM | >50 μmol |
| 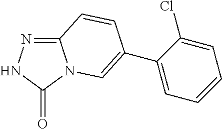 [SMILES: O=c1[nH]nc2ccc(-c3ccccc3Cl)cn12] | Kᵢ = 3.2 μM | >50 μmol |
| 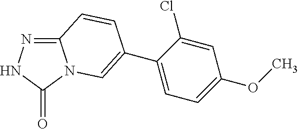 [SMILES: COc1ccc(-c2ccc3n[nH]c(=O)n3c2)c(Cl)c1] | Kᵢ = 2.76 μM | >50 μmol |
| 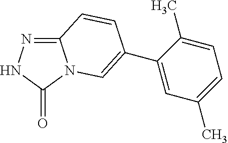 [SMILES: Cc1ccc(C)c(-c2ccc3n[nH]c(=O)n3c2)c1] | Kᵢ = 23.17 μM | >50 μmol |
| 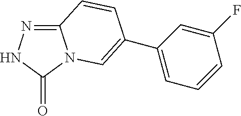 [SMILES: O=c1[nH]nc2ccc(-c3cccc(F)c3)cn12] | Kᵢ = 35.3 μM | >50 μmol |
| 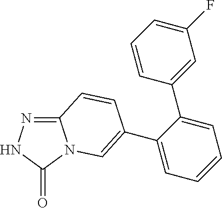 [SMILES: O=c1[nH]nc2ccc(-c3ccccc3-c3cccc(F)c3)cn12] | >50 μmol | >50 μmol |
| 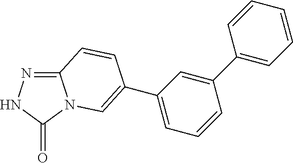 [SMILES: O=c1[nH]nc2ccc(-c3cccc(-c4ccccc4)c3)cn12] | Kᵢ = 27.4 μM | >50 μmol |
| 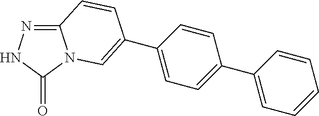 [SMILES: O=c1[nH]nc2ccc(-c3ccc(-c4ccccc4)cc3)cn12] | Kᵢ = 170.4 μM | >50 μmol |
| 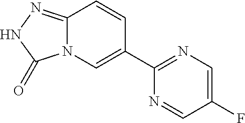 [SMILES: O=c1[nH]nc2ccc(-c3ncc(F)cn3)cn12] | Kᵢ = 104.6 μM | n. d. |
| 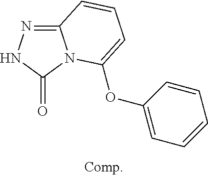 [SMILES: O=c1[nH]nc2cccc(Oc3ccccc3)n12] | >50 μmol | >50 μmol |
| 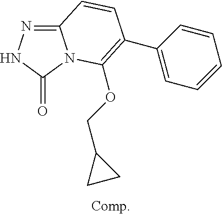 [SMILES: O=c1[nH]nc2ccc(-c3ccccc3)c(OCC3CC3)n12] | >50 μmol | >50 μmol |
| 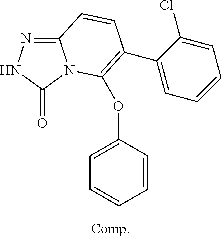 [SMILES: O=c1[nH]nc2ccc(-c3ccccc3Cl)c(Oc3ccccc3)n12] | >50 μmol | >50 μmol |
| 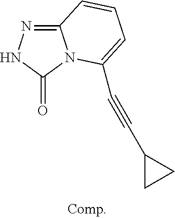 [SMILES: O=c1[nH]nc2cccc(C#CC3CC3)n12] | >50 μmol | >50 μmol |
| 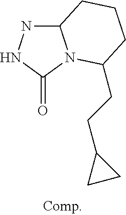 [SMILES: O=C1NNC2CCCC(CCC3CC3)N12] | >50 μmol | >50 μmol |

### General Procedure of CAP-004 Series

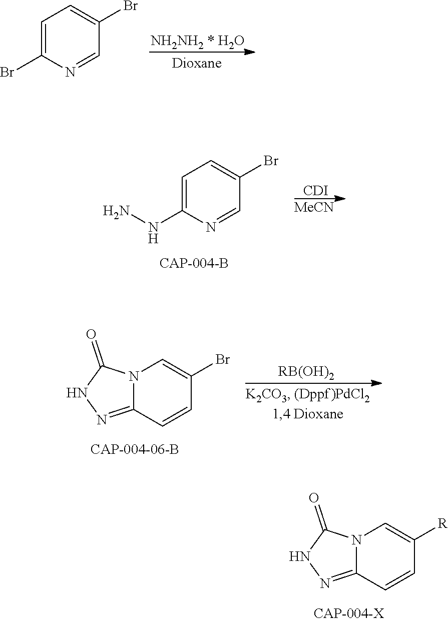

### Synthesis of CAP-004-B

A mixture of 2,5-dibromopyridine (10.00 g, 42.55 mmol) and hydrazine hydrate (85%, 10 mL) in dioxane (100 mL) was refluxed overnight. The solvent was removed and the residue was diluted with water (100 mL). The resultant was extracted with ethyl acetate (50 mL×3), washed with brine (30 mL×3), dried over NaSO₄ and concentrated to give CAP-004-B as a crude product.

### Synthesis of CAP-004-06-B

A mixture of CAP-004-B (crude product form last step) and 1,1′-carbonyldiimidazol (8.27 g, 51.06 mmol) in MeCN (100 mL) was refluxed for 2 h. The mixture was cooled to room temperature and the precipitate was collected by filtration to give CAP-004-C (6.30 g, 70%, two steps) as a white solid.

### General Procedures for the Synthesis of CAP-004-X Series

A mixture of CAP-004-C (0.10 g, 0.47 mmol), RB(OH)₂ (0.56 mmol), K₂CO₃ (0.13 g, 0.94 mmol) and (1,1′-bis(diphenylphosphanyl)ferrocene)PdCl₂ (0.017 g, 0.02 mmol) in dioxane (3 mL) was refluxed for 5 h under N₂ atmosphere. The solvent was removed under reduced pressure and the residue was diluted with water (10 mL). The resultant was extracted with ethyl acetate (5 mL×3), washed with brine (5 mL×3), dried over NaSO₄ and concentrated. The residue was purified by preparative TLC and then purified by preparative HPLC to give CAP-004-X.

### 7-Phenyl[1,2,4]triazolo[4,3-a]pyridin-3(2H)-one (CAP-004-01)

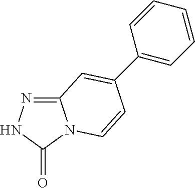 [SMILES: O=c1[nH]nc2cc(-c3ccccc3)ccn12]

CAP-004-01 was obtained as a pale white solid.

Yield: 2%

MS (ESI): 212 (M+H)⁺

¹H NMR (CDCl₃, 400 MHz):

δ=7.84 (d, J=7.2 Hz, 1H), 7.60 (d, J=6.8 Hz, 2H), 7.47-7.50 (m, 3H), 7.28 (s, 1H), 6.82 (t, J=7.2 Hz, 1H).

### 6-Bromo-[1,2,4]triazolo[4,3-a]pyridin-3(2H)-one

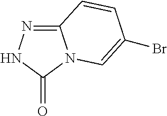 [SMILES: O=c1[nH]nc2ccc(Br)cn12]

CAP-004-06-B was obtained as a pale yellow solid.

Yield: 50%

MS (ESI): 214(M+H)⁺

¹H NMR (CDCl₃, 400 MHz):

δ=10.11 (br, s, 1H), 7.95 (s, 1H), 7.05-7.16 (m, 2H).

### 6-Phenyl-[1,2,4]triazolo[4,3-a]pyridin-3(2H)-one (CAP-004-06)

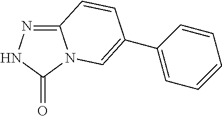 [SMILES: O=c1[nH]nc2ccc(-c3ccccc3)cn12]

CAP-004-06 was obtained as a white solid.

Yield: 1%

MS (ESI): 212 (M+H)⁺

¹H NMR (CDCl₃, 400 MHz):

δ=7.98 (s, 1H), 7.41-7.55 (m, 6H), 7.23(d, J=10.4 Hz, 1H).

### 6-(2-Chlorophenyl)-[1,2,4]triazolo[4,3-a]pyridin-3(2H)-one (CAP-004-07)

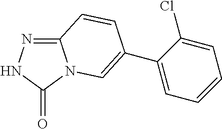 [SMILES: O=c1[nH]nc2ccc(-c3ccccc3Cl)cn12]

CAP-004-07 was obtained as a white solid.

Yield: 3%

MS (ESI): 246 (M+H)⁺

¹H NMR (d₆-DMSO, 400 MHz):

δ=12.60 (s, 1H), 7.82 (t, J=1.2 Hz, 1H), 7.59-7.61 (m, 1H), 7.52-7.53 (m, 1H), 7.44-7.47 (m, 2H), 7.29 (d, J=1.2 Hz, 2H).

### 6-(3-Chlorophenyl)-[1,2,4]triazolo[4,3-a]pyridin-3(2H)-one (CAP-004-08)

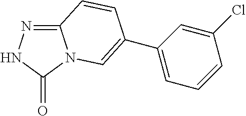 [SMILES: O=c1[nH]nc2ccc(-c3cccc(Cl)c3)cn12]

CAP-004-08 was obtained as a white solid according to the general procedure.

Yield: 5%

MS (ESI): 246 (M+H)⁺

¹H NMR (d₆-DMSO, 400 MHz):

δ=12.58 (s, 1H), 8.13 (t, J=1.2 Hz, 1H), 7.82-7.83 (m, 1H), 7.68-7.71 (m, 1H), 7.60-7.63 (m, 1H), 7.35-7.60 (m, 2H), 7.32 (d, J=0.8 Hz, 1H).

### 6-(4-Chlorophenyl)-[1,2,4]triazolo[4,3-a]pyridin-3(2H)-one (CAP-004-09)

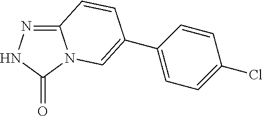 [SMILES: O=c1[nH]nc2ccc(-c3ccc(Cl)cc3)cn12]

CAP-004-09 was obtained as a white solid according to the general procedure.

Yield: 1%

MS (ESI): 246 (M+H)⁺

¹H NMR (d₆-DMSO, 400 MHz):

δ=12.57 (s, 1H), 8.08-8.09 (m, 1H), 7.74-7.77 (m, 2H), 7.57-7.60 (m, 1H), 7.51-7.53 (m, 2H), 7.33-7.36 (m, 1H).

### 6-(2-Chloro-4-methoxyphenyl)-[1,2,4]triazolo[4,3-a]pyridin-3(2H)-one (CAP-004-10)

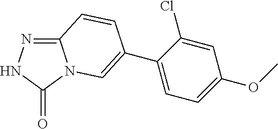 [SMILES: COc1ccc(-c2ccc3n[nH]c(=O)n3c2)c(Cl)c1]

CAP-004-10 was obtained as a white solid according to the general procedure.

Yield: 1%

MS (ESI): 276 (M+H)⁺

¹H NMR (d₆-DMSO, 400 MHz):

δ=12.57 (s, 1H), 7.75 (s, 1H), 7.44 (d, J=8.4 Hz, 1H), 7.26-7.27 (m, 2H), 7.18 (d, J=2.8 Hz, 1H),7.01-7.04 (m, 1H), 3.83 (s, 3H).

### 6-(2,3-Difluorophenyl)-[1,2,4]triazolo[4,3-a]pyridin-3(2H)-one (CAP-004-11)

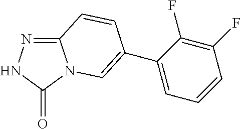 [SMILES: O=c1[nH]nc2ccc(-c3cccc(F)c3F)cn12]

CAP-004-11 was obtained as a white solid according to the general procedure.

Yield: 1%

MS (ESI): 248 (M+H)⁺

¹H NMR (d₆-DMSO, 400 MHz):

δ=12.62 (s, 1H), 8.02 (s, 1H), 7.42-7.50 (m, 3H), 7.31-7.37 (m, 2H).

### 6-(2,5-Dimethylphenyl)-[1,2,4]triazolo[4,3-a]pyridin-3(2H)-one (CAP-004-12)

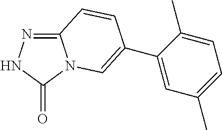 [SMILES: Cc1ccc(C)c(-c2ccc3n[nH]c(=O)n3c2)c1]

CAP-004-12 was obtained as a pale yellow solid according to the general procedure.

Yield: 1%

MS (ESI): 240 (M+H)⁺

¹H NMR (d₆-DMSO, 400 MHz):

δ=12.55 (s, 1H), 7.64 (s, 1H), 7.19-7.28 (m, 3H), 7.11-7.13 (m, 2H), 2.30 (s, 3H), 2.22 (s, 3H).

### 6-(Naphthalen-1-yl)-[1,2,4]triazolo[4,3-a]pyridin-3(2H)-one (CAP-004-13)

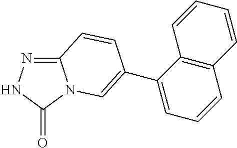 [SMILES: O=c1[nH]nc2ccc(-c3cccc4ccccc34)cn12]

CAP-004-13 was obtained as a pale yellow solid according to the general procedure.

Yield: 1%

MS (ESI): 262 (M+H)⁺

¹H NMR (d₆-DMSO, 400 MHz):

δ=12.61 (s, 1H), 8.00-8.04 (m, 2H), 7.81-7.84 (m, 2H), 7.54-7.60 (m, 4H), 7.33-7.35 (m, 2H).

### 6-(Biphenyl-2-yl)-[1,2,4]triazolo[4,3-a]pyridin-3(2H)-one (CAP-004-22)

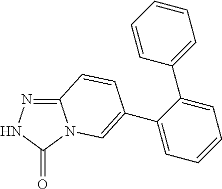 [SMILES: O=c1[nH]nc2ccc(-c3ccccc3-c3ccccc3)cn12]

CAP-004-22 was obtained as a white solid according to the general procedure.

Yield: 1%

MS (ESI): 288 (M+H)⁺

¹H NMR (d₆-DMSO, 400 MHz):

δ=12.50 (m, 1H), 7.64 (s, 1H), 7.43-7.64 (m, 4H), 7.26-7.34 (m, 5H), 6.99 (d, J=0.8 Hz, 1H), 6.64-6.67 (m, 1H).

### 6-(3′-Fluorobiphenyl-2-yl)-[1,2,4]triazolo[4,3-a]pyridin-3(2H)-one (CAP-004-23)

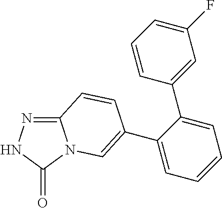 [SMILES: O=c1[nH]nc2ccc(-c3ccccc3-c3cccc(F)c3)cn12]

CAP-004-23 was obtained as a pale white solid according to the general procedure.

Yield: 1%

MS (ESI): 306 (M+H)⁺

¹H NMR (CD₃OD, 400 MHz):

δ=7.73 (t, J=1.2 Hz, 1H), 7.49-7.53 (m, 4H), 7.31-7.36 (m, 1H), 6.97-7.08(m, 4H), 6.84 (dd, J=10 Hz, 1.6Hz, 1H).

### 6-(3-Fluorophenyl)-[1,2,4]triazolo[4,3-a]pyridin-3(2H)-one (CAP-004-23-2)

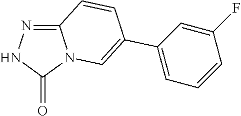 [SMILES: O=c1[nH]nc2ccc(-c3cccc(F)c3)cn12]

CAP-004-23-2 was obtained as a white solid according to the general procedure.

Yield: 1%

MS (ESI): 230 (M+H)⁺

¹H NMR (d₆-DMSO, 400 MHz):

δ=8.09 (t, J=1.2 Hz, 1H), 7.61 (dd, J=9.6 Hz, 1.6 Hz, 1H), 7.42-7.55 (m, 3H), 7.32 (dd, J=9.6 Hz, 0.8 Hz, 1H), 7.13-7.19 (m, 1H).

### 6-(2-(Pyridin-4-yl)phenyl)-[1,2,4]triazolo[4,3-a]pyridin-3(2H)-one (CAP-004-25)

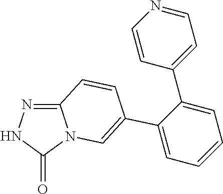 [SMILES: O=c1[nH]nc2ccc(-c3ccccc3-c3ccncc3)cn12]

CAP-004-25 was obtained as a pale yellow solid according to the general procedure.

Yield: 1%

MS (ESI): 289 (M+H)⁺

¹H NMR (CD₃OD, 400 MHz):

δ=8.75 (br, s, 2H), 7.97 (s, 2H), 7.78 (s, 1H), 7.65-7.74 (m, 4H), 7.10 (dd, J=9.6 Hz, 0.8 Hz, 1H), 7.10 (dd, J=9.6 Hz, 1.6Hz, 1H).

### 6-(Biphenyl-3-yl)-[1,2,4]triazolo[4,3-a]pyridin-3(2H)-one (CAP-004-26)

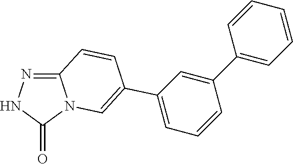 [SMILES: O=c1[nH]nc2ccc(-c3cccc(-c4ccccc4)c3)cn12]

CAP-004-26 was obtained as a pale white solid according to the general procedure.

Yield: 1%

MS (ESI): 288 (M+H)⁺

¹H NMR (d₆-DMSO, 400 MHz):

δ=12.56 (s, 1H), 8.20 (s, 1H), 7.96 (s, 1H), 7.80-7.82 (m, 2H), 7.68-7.72 (m, 3H), 7.54-7.58 (m, 1H), 7.48-7.51 (m, 2H), 7.34-7.42 (m, 2H).

### 6-(2′,6′-Difluorobiphenyl-3-yl)-[1,2,4]triazolo[4,3-a]pyridin-3(2H)-one (CAP-004-27)

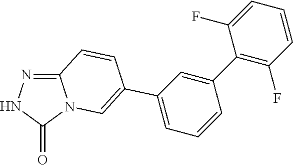 [SMILES: O=c1[nH]nc2ccc(-c3cccc(-c4c(F)cccc4F)c3)cn12]

CAP-004-27 was obtained as a pale yellow solid according to the general procedure.

Yield: 1%

MS (ESI): 324 (M+H)⁺

¹H NMR (d₆-DMSO, 400 MHz):

δ=12.56 (s, 1H), 8.10 (s, 1H), 7.79-7.82 (m, 2H), 7.58 -7.65 (m, 2H), 7.46-7.53 (m, 2H), 7.28-7.36 (m, 1H), 7.23-7.26 (m, 2H).

### 6-(Biphenyl-4-yl)-[1,2,4]triazolo[4,3-a]pyridin-3(2H)-one (CAP-004-30)

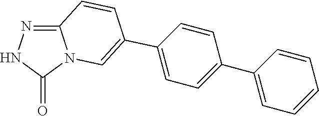 [SMILES: O=c1[nH]nc2ccc(-c3ccc(-c4ccccc4)cc3)cn12]

CAP-004-30 was obtained as a pale white solid according to the general procedure.

Yield: 1%

MS (ESI): 288 (M+H)⁺

¹H NMR (d₆-DMSO, 400 MHz):

δ=12.58 (s, 1H), 8.10 (s, 1H), 7.73-7.83 (m, 6H), 7.64-7.68 (m, 1H), 7.41-7.51 (m, 2H), 7.35-7.39(m, 2H).

### 6-(5-Fluoropyrimidin-2-yl)-[1,2,4]triazolo[4,3-a]pyridin-3(2H)-one (CAP-004-38)

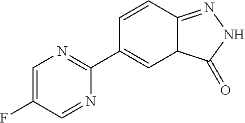 [SMILES: O=C1NN=C2C=CC(c3ncc(F)cn3)=CC12]

CAP-004-38 was obtained as a pale yellow solid according to the general procedure.

Yield: 1%

MS (ESI): 232 (M+H)⁺

¹H NMR (CD₃OD, 400 MHz):

δ=8.87(s, 1H), 8.82 (s, 2H), 8.22 (dd, J=10 Hz, 1.6 Hz, 1H), 7.31 (d, J=6 Hz, 1H)

### Synthesis of CAP-004-04

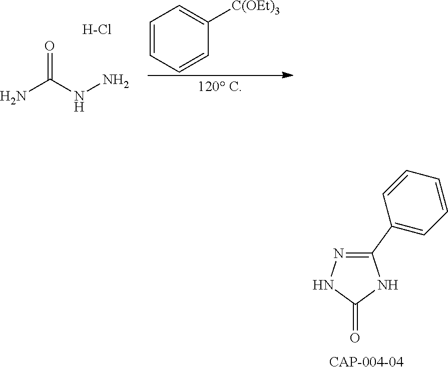

A mixture of hydrazinecarboxamide hydrochloride (0.22 g, 1.96 mmol) and (triethoxymethyl) benzene (0.67 g, 2.95 mmol) was stirred at 120° C. for overnight. The crude product was purified by preparative TLC and then purified by preparative HPLC to give CAP-004-04 (0.017 g, 13%) as a white solid.

### 3-Phenyl-1H-1,2,4-triazol-5(4H)-one (CAP-004-04)

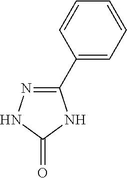 [SMILES: O=c1[nH]nc(-c2ccccc2)[nH]1]

CAP-004-04 was obtained as a white solid.

Yield: 13%

MS (ESI): 162 (M+H)⁺

¹H NMR (d₆-DMSO, 400 MHz):

δ=12.02 (s, 1H), 11.67 (s, 1H), 7.78 (d, J=8.0 Hz, 2H), 7.45-7.49 (m, 3H).

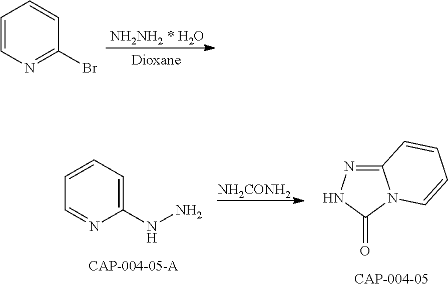 [SMILES: Brc1ccccn1 ; NNc1ccccn1 ; "NH2CONH2" ; O=c1[nH]nc2ccccn12]

### Synthesis of CAP-004-05-A

A mixture of 2-bromopyridine (1.00 g, 6.37 mmol) and hydrazine hydrate (85%, 10 mL) in dioxane (20 mL) was refluxed overnight. The solvent was removed to give CAP-004-05-A as a crude product.

### Synthesis of CAP-004-05:

A mixture of crude CAP-004-05-A and urea (1.70 g, 27.50 mmol) was stirred at 130° C. for 3 h. The resultant was purified by column chromatography on silica gel (ethyl acetate/petroleum ether=1/10-3/1) to give CAP-004-05 (0.13 g, 15%, two steps) as a pale white solid.

### [1,2,4]Triazolo[4,3-a]pyridin-3(2H)-one (CAP-004-05)

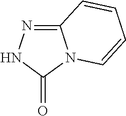

CAP-004-05 was obtained as a pale white solid.

Yield: 15%

MS (ESI): 136 (M+H)⁺

¹H NMR (CDCl₃, 400 MHz):

δ=7.78 (d, J=7.2 Hz, 1H), 7.13 (s, 1H), 7.12 (d, J=4.4 Hz, 1H), 6.49-6.53 (m, 1H) ¹³C NMR (d₆-DMSO, 300 MHz)

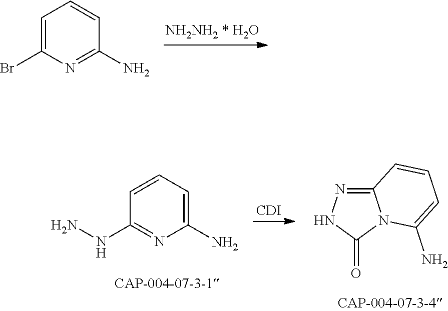

### Synthesis of CAP-004-07-3-1″:

A mixture of 6-bromopyridin-2-amine (1.00 g, 5.81 mmol) and hydrazine hydrate (85%, 2 mL) in dioxane (20 mL) was refluxed overnight. The solvent was removed to give CAP-004-07-3-1″ as a crude product.

### Synthesis of CAP-004-07-3-4″:

A mixture of CAP-004-07-3-1″ (crude product form last step) and 1,1′-carbonyldiimidazol (1.13 g, 6.98 mmol) in MeCN (50 mL) was refluxed for 2 h. The mixture was cooled to room temperature and the precipitate was collected by filtration and purified by preparative HPLC to give CAP-004-07-3-4″ (0.017 g, 2%, two steps) as a brown solid.

### 5-Amino-[1,2,4]triazolo[4,3-a]pyridin-3(2H)-one (CAP-004-07-3-4″)

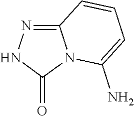 [SMILES: Nc1cccc2n[nH]c(=O)n12]

CAP-004-07-3-4″ was obtained as a brown solid.

Yield: 2%

MS (ESI): 151 (M+H)⁺

¹H NMR (CD₃OD, 400 MHz):

δ=7.00 (t, J=4.8 Hz, 1H), 6.22 (d, J=9.2 Hz, 1H), 5.38 (d, J=7.2 Hz, 1H).

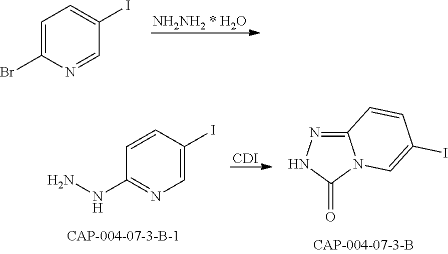

### 6-lodo-[1,2,4]triazolo[4,3-a]pyridin-3(2H)-one (CAP-004-07-3-B)

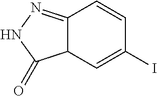 [SMILES: O=C1NN=C2C=CC(I)=CC12]

CAP-004-07-3-B was obtained as a yellow solid.

Yield: 26%

MS (ESI): 262 (M+H)⁺

¹H NMR (d₆-DMSO, 400 MHz):

δ=12.55 (s, 1H), 8.06 (s, 1H), 7.30 (dd, J=9.6 Hz, 4.6 Hz, 1H), 7.08 (dd, J=9.6 Hz, 0.8 Hz, 1 H).

### 6-Bromo-[1,2,4]triazolo[4,3-a]pyridin-3(2H)-one (CAP-004-06-B)

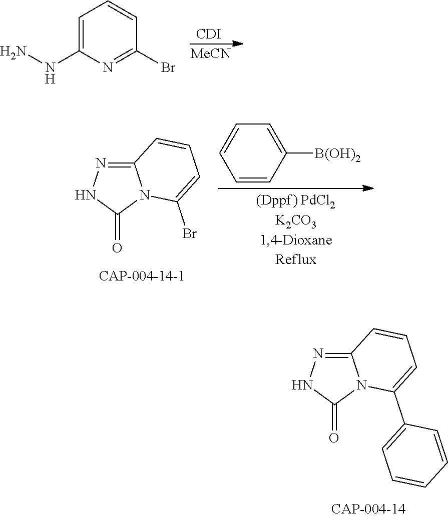

### 5-Phenyl-[1,2,4]triazolo[4,3-a]pyridin-3(2H)-one (CAP-004-14)

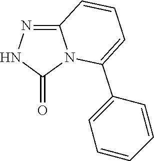 [SMILES: O=c1[nH]nc2cccc(-c3ccccc3)n12]

CAP-004-14 was obtained as a white solid according to the general procedure.

Yield: 15%

MS (ESI): 212 (M+H)⁺

¹H NMR (d₆-DMSO, 400 MHz):

δ=12.40 (s, 1H), 7.40-7.49 (m, 5H), 7.16-7.18 (m, 2H), 6.36-6.38 (m, 1H).

### 5-(Pyrimidin-2-yl)-[1,2,4]triazolo[4,3-a]pyridin-3(2H)-one (CAP-004-44)

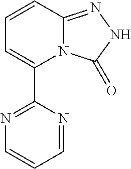 [SMILES: O=c1[nH]nc2cccc(-c3ncccn3)n12]

CAP-004-44 was obtained as a pale yellow solid according to the general procedure.

Yield: 10%

MS (ESI): 214 (M+H)⁺

¹H NMR (CD₃OD, 400 MHz):

δ=8.91 (d, J=4.8 Hz, 2H), 7.57 (t, J=4.8 Hz, 1H), 7.32 (t, J=2.4 Hz, 2H), 6.78-6.80 (m, 1H).

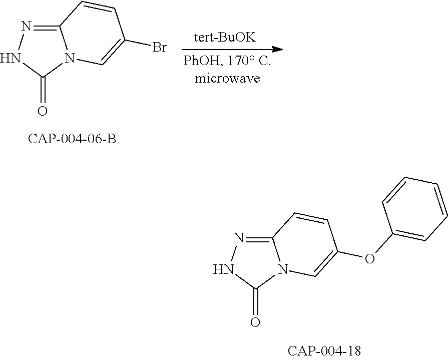 [SMILES: O=c1[nH]nc2ccc(Br)cn12 ; O=c1[nH]nc2ccc(Oc3ccccc3)cn12]

### Synthesis of CAP-004-18:

CAP-004-06-B (100 mg, 0.47 mmol) and t-BuOK (105 mg, 0.93 mmol) were added to phenol (2 mL). This reaction mixture was stirred for 2 h at 170° C. in a microwave. This mixture was filtered and purified by preparative HPLC to afford CAP-004-18 (6 mg, 5%) as a white solid.

### 6-Phenoxy-[1,2,4]triazolo[4,3-a]pyridin-3(2H)-one (CAP-004-18)

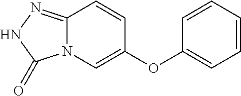 [SMILES: O=c1[nH]nc2ccc(Oc3ccccc3)cn12]

Pale white solid

Yield: 1%

MS (ESI): 228.1 (M+H)⁺

¹H NMR (CD₃OD, 400 MHz):

δ=7.63 (d, J=6.0 Hz, 1H), 7.47 (dd, J=7.6 Hz, 8.4 Hz, 2H), 7.28 (t, J=7.6 Hz, 1H), 7.20 (dd, J=1.2 Hz, 8.8 Hz, 2H), 6.57 (t, J=7.2 Hz, 1H), 6.48 (d, J=7.2 Hz, 1H), 5-Phenoxy-[1,2,4]triazolo[4,3-a]pyridin-3(2H)-one (CAP-004-36)

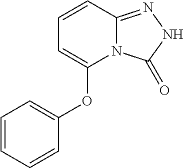 [SMILES: O=c1[nH]nc2cccc(Oc3ccccc3)n12]

CAP-004-36 was obtained as brown oil starting from CAP-004-14-1 in the same manner as CAP-004-18.

Yield: 8%

MS (ESI): 228 (M+H)⁺

¹H NMR (CD₃OD, 400 MHz):

δ=7.49 (t, J=8.0 Hz, 2H), 7.31 (t, J=7.2 Hz, 1H), 7.25 (d, J=8.4 Hz, 2H), 7.03-7.07 (m, 1H), 6.80 (d, J=9.6 Hz, 1H), 5.53 (d, J=7.2 Hz, 1H).

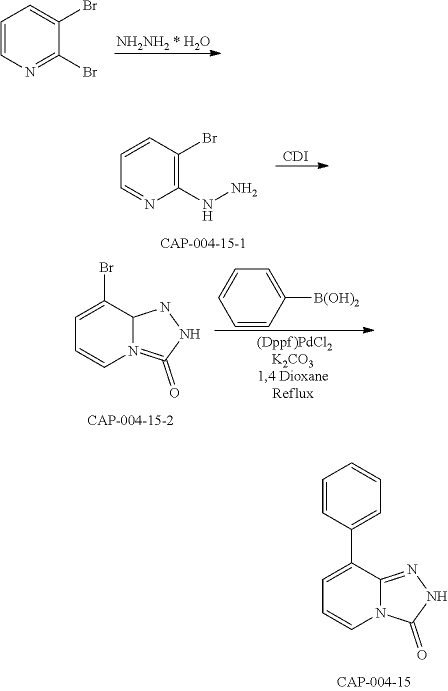

### 8-Phenyl-[1,2,4]triazolo[4,3-a]pyridin-3(2H)-one (CAP-004-15)

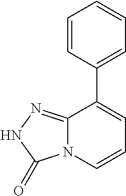 [SMILES: O=c1[nH]nc2c(-c3ccccc3)cccn12]

CAP-004-15 was obtained as a pale yellow solid according to the general procedure.

Yield: 8%

MS (ESI): 212 (M+H)⁺

¹H NMR (d₆-DMSO, 400 MHz):

δ=12.63 (s, 1H), 7.96-7.98 (m, 2H), 7.85 (dd, J=7.2 Hz, 0.8Hz, 1H), 7.44-7.52 (m, 4H), 6.71 (t, J=6.8 Hz, 1H).

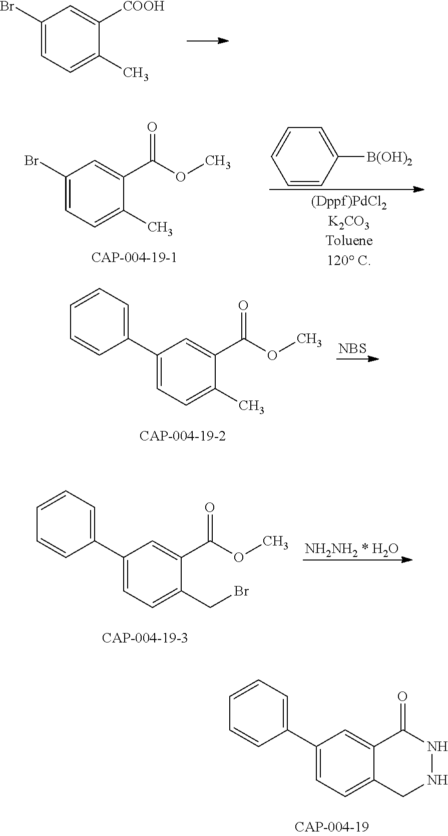

### Synthesis of CAP-004-19-1:

A mixture of 5-bromo-2-methylbenzoic acid (1.00 g, 4.7 mmol) and thionyl chloride (1.40 g, 11.6 mmol) in methanol (30 mL) was refluxed for 2 h. The solvent was removed under reduced pressure and the pH of the residue was adjusted to 7. The resultant was extracted with ethyl acetate. The organic phase was dried over anhydrous Na₂SO₄, filtered and concentrated to give CAP-004-19-1 (1.02 g, 95%) as a pale yellow solid.

### Synthesis of CAP-004-19-2:

A mixture of CAP-004-19-1 (1.00 g, 4.3 mmol), phenylboronic acid (0.60 g, 5.2 mmol), K₂CO₃ (1.21 g, 8.7 mmol) and (1,1′-bis(diphenylphosphanyl)ferrocene)₂PdCl₂ (0.20 g, 0.26 mmol) in dioxane (50 mL) was refluxed for 5 h under N₂ atmosphere. The solvent was removed under reduced pressure and the residue was purified by column chromatography on silica gel (ethyl acetate/petroleum ether=1/10˜1/1) to give CAP-004-19-3 (0.80 g, 82%) as a white solid.

### Synthesis of CAP-004-19-3

A mixture of CAP-004-19-2 (0.80 g, 3.5 mmol) and NBS (0.76 g, 4.2 mmol) in chloroform (30 mL) was refluxed overnight. The solvent was removed and petroleum ether (50 ml) was added. The formed precipitate was removed by filtration and the filtrate was concentrated to give CAP-004-19-3 (0.71 g, 64%) as a colorless oil.

### Synthesis of CAP-004-19:

A mixture of CAP-004-19-3 (0.20 g, 0.66 mmol) and hydrazine hydrate (85%, 2 mL) in dioxane (10 mL) was refluxed overnight. The solvent was removed and the residue was purified by preparative HPLC to give CAP-004-19 (0.040 g, 28%)) as a white solid.

### 7-Phenyl-3,4-dihydrophthalazin-1(2H)-one (CAP-004-19)

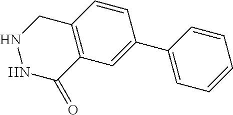 [SMILES: O=C1NNCc2ccc(-c3ccccc3)cc21]

CAP-004-19 was obtained as a white solid according to the general procedure.

Yield: 5%

MS (ESI): 225 (M+H)⁺

¹H NMR (d₆-DMSO, 400 MHz):

δ=7.89-7.91 (m, 2H), 7.73 (d, J=7.2 Hz, 2H), 7.64 (d, J=8.4 Hz, 1 H), 7.49 (t, J=7.6 Hz, 2H), 7.40 (t, J=7.2 Hz, 1H), 4.54 (s, 2H)

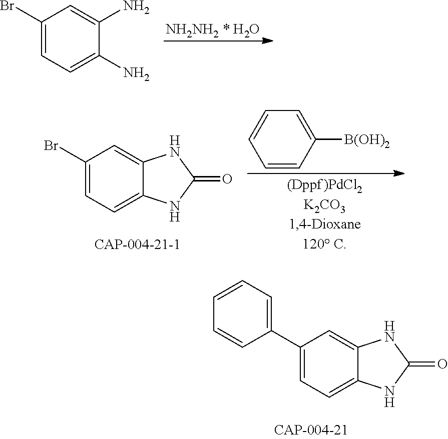

### Synthesis of CAP-004-21-1

A mixture of 4-bromobenzene-1,2-diamine (1.00 g, 5.3 mmol) and urea (0.97 g, 15.0 mmol) was stirred at 130° C. for overnight. The residue was purified by column chromatography on silica gel (ethyl acetate/petroleum ether=1/10˜3/1) to give CAP-004-21-1 (0.31 g, 27%) as a pale yellow solid.

### Synthesis of CAP-004-21

A mixture of CAP-004-21-1 (0.15 g, 0.71 mmol), phenylboronic acid (0.13 g, 1.1 mmol), K₂CO₃ (0.20 g, 1.4 mmol) and (1,1′-bis(diphenylphosphanyl)ferrocene)PdCl₂ (0.037 g, 0.04 mmol) in dioxane (20 mL) was refluxed for 5 h under N₂ atmosphere. The solvent was removed under reduced pressure and the residue was purified by preparative HPLC to give CAP-004-21 (0.059 g, 40%) as a white solid.

### 5-Phenyl-1H-benzo[d]imidazol-2(3H)-one (CAP-004-21)

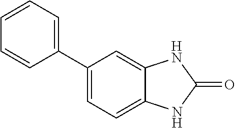 [SMILES: O=c1[nH]c2ccc(-c3ccccc3)cc2[nH]1]

CAP-004-21 was obtained as a pale yellow solid according to the general procedure.

Yield: 10%

MS (ESI): 211 (M+H)⁺

¹H NMR (d₆-DMSO, 400 MHz):

δ=10.70 (d, J=8.4 Hz, 2H), 7.58 (d, J=7.2 Hz, 2H), 7.41-7.45 (m, 2H), 7.29-7.32 (m, 1H), 7.21-7.24 (m, 1H), 7.14 (d, J=1.2 Hz, 1H), 7.00 (d, J=8.0 Hz, 1H).

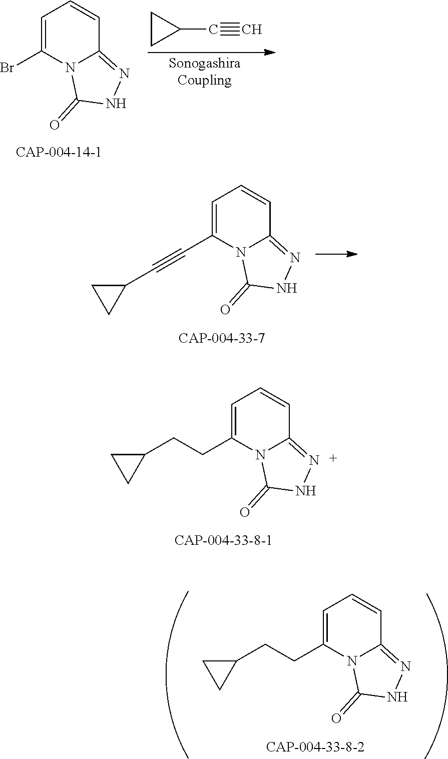

### Synthesis of CAP-004-33-7:

To a solution of CAP-004-14-1 (100.0 mg, 0.47 mmol) in 10 mL of 1,4-dioxane was added Cul (9.0 mg, 0.05 mmol), Et₃N (95.2 mg, 0.94 mmol) and (PPh₃)₂PdCl₂ (13.3 mg, 0.02 mmol) under nitrogen. The mixture was stirred at room temperature for 0.5 h and ethynylcyclopropane (46.5 mg, 0.70 mmol) was then added. The resulting mixture was continued to stir at room temperature for 5 h. After cooling to room temperature, the mixture was filtered and the filtrate was concentrated in vacuo. The residue was purified by column chromatography on silica gel (methanol : dichloromethane=1/100-1/10) to afford CAP-004-33-7 (19.0 mg, 20%) as a pale yellow solid.

### Synthesis of CAP-004-33-8-1 and CAP-004-33-8-2

CAP-004-33-7 (30.0 mg, 0.15 mmol) and palladium on activated carbon (5% Pd, 3.0 mg) were mixed with methanol (10 mL) and attached to a hydrogenation apparatus. The system was evacuated and then refilled with hydrogen. The reaction mixture was stirred at 40° C. overnight. After cooling to room temperature, the mixture was filtered and the filtrate was concentrated in vacuo. The residue was purified by preparative HPLC to afford CAP-004-33-8-1 (9.4 mg) and CAP-004-33-8-2 (10.2 mg) as colorless oils.

### 5-(Cyclopropylethynyl)-[1,2,4]triazolo[4,3-a]pyridin-3(2H)-one (CAP-004-33-7)

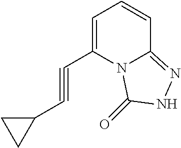 [SMILES: O=c1[nH]nc2cccc(C#CC3CC3)n12]

CAP-004-33-7 was obtained as a pale yellow solid according to the general procedure.

Yield: 20%

MS (ESI): 200 (M+H)⁺

¹H NMR (d₆-DMSO, 400 MHz):

δ=6.77 (t, J=3.2 Hz, 2H), 5.89 (s, 1H), 5.80-5.83 (m, 1H), 1.74-1.76 (m, 1H), 0.87-0.92 (m, 4H).

### 5-(2-Cyclopropylethyl)-5,6,7,8-tetrahydro-[1,2,4]triazolo[4,3-a]pyridin-3(2H)-one (CAP-004-33-8-1)

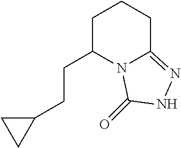 [SMILES: O=c1[nH]nc2n1C(CCC1CC1)CCC2]

CAP-004-33-8-1 was obtained as colorless oil according to the general procedure.

Yield: 6%

MS (ESI): 208 (M+H)⁺

¹H NMR (CD₃OD, 400 MHz):

δ=3.89-3.93 (m, 1H), 2.54-2.63 (m, 2H), 2.05-2.08 (m, 1H), 1.63-1.88 (m, 5H), 1.19-1.25 (m, 2H), 0.65-0.66 (m, 1H), 0.37-0.39 (m, 2H), 0.01-0.02 (m, 2H).

### 5-(2-Cyclopropylethyl)hexahydro-[1,2,4]triazolo[4,3-a]pyridin-3(5H)-one (CAP-004-33-8-2)

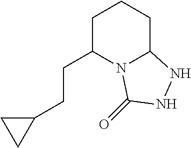 [SMILES: O=C1NNC2CCCC(CCC3CC3)N12]

CAP-004-33-8-2 was obtained as colorless oil according to the general procedure.

Yield: 8%

MS (ESI): 210 (M+H)⁺

¹H NMR (CD₃OD, 400 MHz):

δ=3.88-3.92 (m, 1H), 2.55-2.65 (m, 2H), 1.74-1.98 (m, 5H), 1.54-1.60 (m, 1H), 1.25-1.37 (m, 6H), 0.89 (t, J=6.4 Hz, 2H).

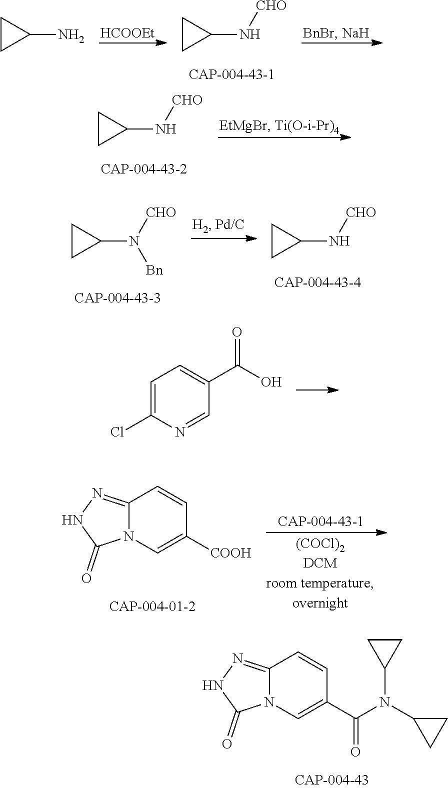

### Synthesis of CAP-004-43-1:

A mixture of cyclopropanamine (5.00 g, 0.088 mol) and ethyl formate (13.00 g, 0.180 mol) was stirred at 50° C. for 5 h. The solvent was removed in vacuo to afford CAP-004-43-1 (7.02 g, 84%) as a pale yellow oil.

### Synthesis of CAP-004-43-2:

To a mixture of sodium hydride (13.70 g, 0.36 mol) and CAP-004-43-1 (15.21 g, 0.18 mol) in 100 mL of anhydrous benzene was added benzyl bromide (29.7 mL, 0.25 mol). The mixture was stirred at room temperature for 10 h. Water (200 ml) was added and the resultant was extracted with ethyl acetate. The organic phase was dried and concentrated to give CAP-004-43-2 (28.21 g, 90%) as a colorless oil.

### Synthesis of CAP-004-43-3:

To a mixture of CAP-004-43-2 (0.50 g, 2.9 mmol) and Ti(O-i-Pr)₄ (0.81 g, 3.7 mmol) in THF (50 mL) was added EtMgBr (0.87 g, 6.6 mmol) at 0° C. The solution was stirred at room temperature overnight. Water (50 mL) was added and the resultant was extracted with ethyl acetate. The organic phase was dried and concentrated to give CAP-004-43-3 (0.50 g, 94%) as a colorless oil.

### Synthesis of CAP-004-43-4

A solution of CAP-004-43-3 (1.73 g, 9.2 mmol) in 40 mL of anhydrous methanol was hydrogenated over 100 mg of 10% Pd on charcoal at atmospheric pressure. The mixture was stirred at 40° C. for 4 h. After cooling to room temperature, the mixture was filtered and the pH of the filtrate was adjusted to 3. The solvent was removed in vacuo to give CAP-004-43-4 (1.10 g, 88%) as a white solid.

### Synthesis of DUAL-004-01-2

Into a 50 mL 3-neck flask were added 6-chloronicotinic acid (1.00 g, 5.0 mmol), ethyl hydrazinecarboxylate (0.67 g, 6.5 mmol), water (100 mL) and isopropanol (5 mL) successively at room temperature. The resulting suspension was heated to 95° C. and stirred for 17 h while stirring gently. The reaction was cooled to room temperature to give a solid. 2N NaOH (5 mL, 10.0 mmol) was added over 10 min. The pH was adjusted to 8.4 by addition of concentrated hydrochloric acid. The reaction mixture was then heated back to 94° C. and stirred at this temperature for about 48 h. The mixture was then cooled to about 46° C. and concentrated hydrochloric acid was added over 1-2 minutes to give pH=˜2. The reaction mixture was cooled to room temperature and then filtered. The filter cake was rinsed with water (3×10 mL) and dried to afford DUAL-004-01-2 (0.6 g, 55%) as a white solid.

### Synthesis of CAP-004-43:

A mixture of DUAL-004-01-2 (50.0 mg, 0.28 mmol) and oxalyl dichloride (106.0 mg, 0.84 mmol) in dichloromethane (10 mL) was refluxed for 2 h and concentrated in vacuo. To the residue in dichloromethane (10 mL) was added a mixture of CAP-004-43-4 (74.6 mg, 0.56 mmol) and Et₃N (85.0 mg, 0.84 mmol) in dichloromethane (10 mL) at 0° C. The mixture was stirred at room temperature overnight. The solvent was removed in vacuo and the residue was purified by preparative HPLC to give CAP-004-43 (20.1 mg, 27%) as a pale white solid.

### N,N-Dicyclopropyl-3-oxo-2,3-dihydro-[1,2,4]triazolo[4,3-a]pyridine-6-carboxamide (CAP-004-43)

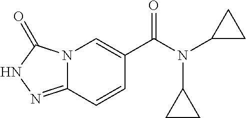 [SMILES: O=C(c1ccc2n[nH]c(=O)n2c1)N(C1CC1)C1CC1]

CAP-004-43 was obtained as a pale white solid according to the general procedure.

Yield: 5%

MS (ESI): 259 (M+H)⁺

¹H NMR (d₆-DMSO, 400 MHz):

δ=12.60 (s, 1H), 8.07 (s, 2H), 7.30 (dd, J=9.6, 1.2 Hz, 1H), 7.21 (d, J=10.0 Hz, 1H), 2.75-2.81 (m, 2H), 0.65-0.74 (m, 8H).

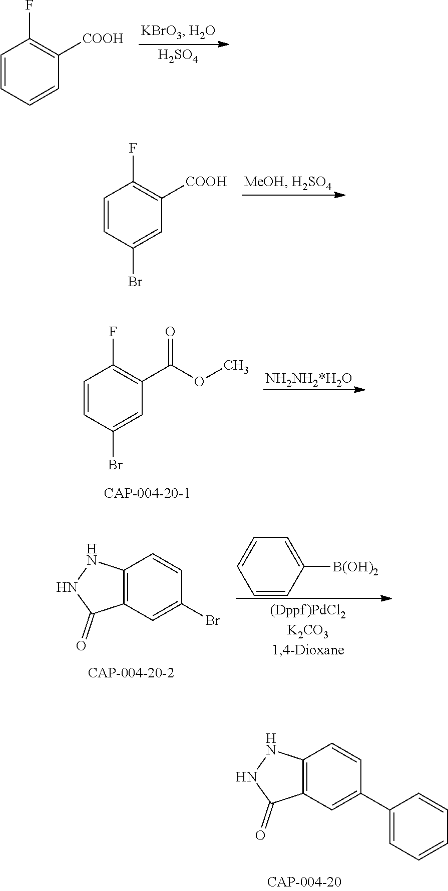

### Synthesis of 5-bromo-2-fluorobenzoic acid

2-Fluorobenzoic acid (1.0 g, 7.14 mmol), KBrO₃ (2.38 g, 14.28 mmol) and water (2 mL) were added to H₂SO₄ (5 mL). This reaction mixture was stirred for 2 h at 90° C. The resulting mixture was extracted with ethyl acetate. The organic layer was dried over Na₂SO₄, filtered and concentrated in vacuo to afford the crude product (1.2 g, 77%) as a white solid.

### Synthesis of CAP-004-20-1

5-Bromo-2-fluorobenzoic acid (1.2 g, 5.48 mmol), and H₂SO₄ (2 mL) were added to methanol (20 mL). This reaction mixture was stirred for 2 h at reflux. The mixture was concentrated in vacuo. The residue was diluted with saturated aqueous NaHCO₃ solution and then extracted with ethyl acetate. The organic layer was dried over Na₂SO₄, filtered and concentrated in vacuo to afford the crude product (1.3 g, 99%) as a yellow oil.

### Synthesis of CAP-004-20-2

CAP-004-20-1 (500 mg, 2.15 mmol) and hydrazine hydrate (2 mL) were added to ethanol (3 mL). This reaction mixture was stirred for 2 h at 100° C. under microwave. The mixture was purified by preparative TLC (petroleum ether/ethyl acetate=1/2) to afford CAP-004-20-2 (200 mg, 43%) as a white solid.

### Synthesis of CAP-004-20

CAP-004-20-2 (110 mg, 0.52 mmol), phenylboronic acid (94 mg, 0.78 mmol), Pd(1,1′-bis(diphenylphosphanyl)ferrocene)Cl₂ (8 mg) and K₂CO₃ (142 mg, 1.03 mmol) were added to 1,4-dioxane (2 mL). This reaction mixture was stirred for 1.5 h at 145° C. under microwave. The mixture was filtered and purified by preparative HPLC to afford CAP-004-20 (12 mg, 11%) as a white solid.

### 5-Phenyl-1 H-indazol-3(2H)-one (CAP-004-20)

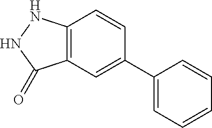 [SMILES: O=c1[nH][nH]c2ccc(-c3ccccc3)cc12]

Pale white solid

Yield: 1%

MS (ESI): 211.2 (M+H)⁺

¹H NMR (CD₃OD, 400 MHz):

δ=7.98 (d, J=0.8 Hz, 1H), 7.79 (dd, J=8.8 Hz, 2.0 Hz, 1H), 7.65 (d, J=7.2 Hz, 2H), 7.46 (t, J=7.2 Hz, 2H), 7.39 (d, J=8.8 Hz, 1H), 7.34 (t, J=7.6 Hz, 1H).

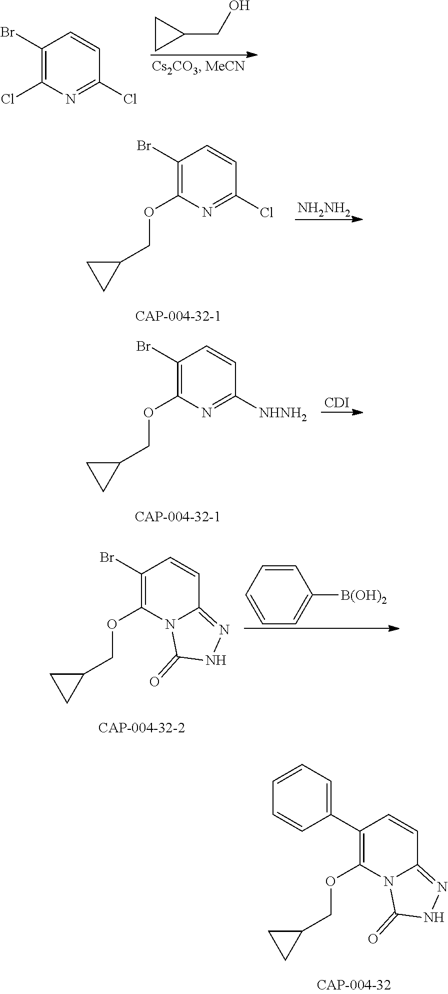

### Synthesis of CAP-004-32-1

3-Bromo-2,6-dichloropyridine (1.04 g, 4.58 mmol), cyclopropylmethanol (1.6 g, 22.91 mmol) and Cs₂CO₃ (2.9 g, 9.16 mmol) were added to CH₃CN (40 mL). This reaction mixture was stirred overnight at 80° C. The mixture was filtered and concentrated in vacuo to afford CAP-004-32-1 (1.05 g, 80%) as a brown oil, which was used to the next step without further purification.

### Synthesis of CAP-004-32-2

CAP-004-32-1 (1.05 g, 4.01 mmol) was added to hydrazine hydrate (10 mL). This reaction mixture was stirred overnight at 90° C. The mixture was concentrated in vacuo. The residue was diluted with water and extracted with ethyl acetate. The organic layer was dried over Na₂SO₄, filtered and concentrated in vacuo. The residue was purified by preparative TLC (petroleum ether/ethyl acetate=1/1) to afford CAP-004-32-2 (300 mg, 30%) as a brown oil.

### Synthesis of CAP-004-32-3

CAP-004-32-2 (300 mg, 1.16 mmol) and 1,1′-carbonyldiimidazol (250 mg, 1.16 mmol) were added to CH₃CN (10 mL). This mixture was stirred for 4 h at 60° C. The mixture was concentrated and purified by preparative TLC (ethyl acetate) to afford CAP-004-32-3 (59 mg, 18%) as a brown solid.

### Synthesis of CAP-004-32

CAP-004-32-3 (35 mg, 0.12 mmol), phenylboronic acid (30 mg, 0.19 mmol), Pd(1,1′-bis(diphenylphosphanyl)ferrocene)Cl₂ (5 mg) and K₂CO₃ (40 mg, 0.25 mmol) were added to 1,4-dioxane (2 mL). This reaction mixture was stirred for 50 min at 140° C. under microwave irratiation. The mixture was concentrated and purified by preparative TLC (petroleum ether/ethyl acetate=1/3) to afford CAP-004-32 (13 mg, 35%) as a white solid.

### 5-(Cyclopropylmethoxy)-6-phenyl-[1,2,4]triazolo[4,3-a]pyridin-3(2H)-one (CAP-004-32)

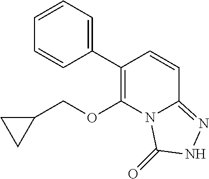 [SMILES: O=c1[nH]nc2ccc(-c3ccccc3)c(OCC3CC3)n12]

Pale white solid

Yield: 1%

MS (ESI): 282.2 (M+H)⁺

¹H NMR (CD₃OD, 400 MHz):

δ=7.79 (d, J=7.6 Hz, 2H), 7.43 (t, J=6.8 Hz, 2H), 7.35 (t, J=7.2 Hz, 1H), 7.25 (d, J=8.0 Hz, 1H), 5.88 (d, J=8.0 Hz, 1H), 4.09 (d, J=6.8 Hz, 2H), 1.43 (m, 1H), 0.66-0.71 (m, 2H), 0.46-0.50 (m, 2H).

### 6-(2-Chlorophenyl)-5-(cyclopropylmethoxy)-[1,2,4]triazolo[4,3-a]pyridin-3(2H)-one (CAP-004-34)

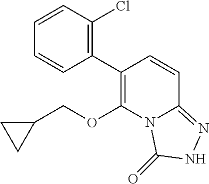 [SMILES: O=c1[nH]nc2ccc(-c3ccccc3Cl)c(OCC3CC3)n12]

Pale white solid

Yield: 1%

MS (ESI): 316.2 (M+H)⁺

¹H NMR (CD₃OD, 400 MHz):

δ=7.51-7.53 (m, 1H), 7.44-7.47 (m, 1H), 7.36-7.41 (m, 2H), 7.05 (d, J=7.6 Hz, 1H), 5.89 (d, J=7.6 Hz, 1H), 4.11 (d, J=7.2 Hz, 2H), 1.39-1.48 (m, 1H), 0.67-0.73 (m, 2H), 0.47-0.51 (m, 2H).

### 6-(2-Chlorophenyl)-5-phenoxy-[1,2,4]triazolo[4,3-a]pyridin-3(2H)-one (CAP-004-37)

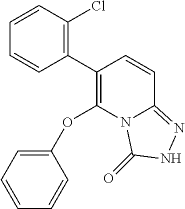 [SMILES: O=c1[nH]nc2ccc(-c3ccccc3Cl)c(Oc3ccccc3)n12]

Pale white solid

Yield: 1%

MS (ESI): 338.1 (M+H)⁺

¹H NMR (CDCl₃, 400 MHz):

δ=7.42-7.53 (m, 4H), 7.27-7.37 (m, 5H), 6.92 (d, J=7.6 Hz, 1H), 5.48 (d, J=7.6 Hz, 1H).

### 6-Bromo-5-phenoxy-[1,2,4]triazolo[4,3-a]pyridin-3(2H)-one (CAP-004-37-3)

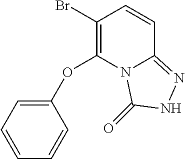 [SMILES: O=c1[nH]nc2ccc(Br)c(Oc3ccccc3)n12]

Pale white solid

Yield: 1%

MS (ESI): 308.0 (M+2H)⁺

¹H NMR (CDCl₃, 400 MHz):

δ=7.46 (t, J=6.8 Hz, 2H), 7.31 (t, J=7.6 Hz, 1H), 7.17-7.22 (m, 3H), 5.38 (d, J=8.0 Hz, 1H).

1. A compound having the general formula (I), optionally in the form of a pharmaceutically acceptable salt, solvate, polymorph, prodrug, tautomer, racemate, codrug, cocrystal, enantiomer, or diastereomer or mixture thereof, wherein the compound is for use in the treatment, amelioration or prevention of a viral disease 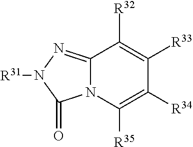
wherein
R³¹ is selected from —H and —C₁₋₆ alkyl;
R³² is selected from —H and —C₁₋₆ alkyl;
R³³ is selected from —H and —C₁₋₆ alkyl;
R³⁴ is selected from —(X³²)ₜ (optionally substituted hydrocarbon group which contains from 5 to 20 carbon atoms and optionally 1 to 4 heteroatoms selected from O, N and S);
R³⁵ is selected from —H and —C₁₋₆ alkyl;
R³⁶ is selected from —H, (optionally substituted C₁₋₆ alkyl), (optionally substituted C₃₋₉ carbocyclyl), —C₁₋₄ alkyl (optionally substituted C₃₋₉ carbocyclyl), (optionally substituted heterocyclyl having 3 to 9 ring atoms), and —C₁₋₄ alkyl (optionally substituted heterocyclyl having 3 to 9 ring atoms);
R³⁸ is selected from —H, (optionally substituted C₁₋₆ alkyl), (optionally substituted C₃₋₉ carbocyclyl), —C₁₋₄ alkyl(optionally substituted C₃₋₉ carbocyclyl), (optionally substituted heterocyclyl having 3 to 9 ring atoms), and —C₁₋₄ alkyl (optionally substituted heterocyclyl having 3 to 9 ring atoms);
X³² is selected from NR³⁶, N(R³⁶)C(O), C(O)NR³⁶, O, C(O), C(O)O, OC(O), N(R³⁶)SO₂, SO₂N(R³⁶), S, SO, and SO₂;
s is 0 to 4; and
t is 0 or 1;
wherein the alkyl group can be optionally substituted with one or more substituents which are independently selected from -halogen, —CN, —CF₃, —OCF₃, —(CH₂)ₛ—X³²—R³⁸, —C₃₋₉ carbocyclyl, and (heterocyclyl having 3 to 9 ring atoms); and
wherein the hydrocarbon group, heterocyclyl group and/or carbocyclyl group can be optionally substituted with one or more substituents which are independently selected from -halogen, —CN, —CF₃, —OCF₃, —(CH₂)ₛ—X³²—R³⁸, —C₁₋₆ alkyl, —C₃₋₉ carbocyclyl which is optionally substituted by —OH or -Hal, —C₁₋₄ alkyl-C₃₋₉ carbocyclyl which is optionally substituted by —OH or -Hal, (heterocyclyl having 3 to 9 ring atoms which is optionally substituted by —OH or -Hal), and —C₁₋₄ alkyl-(heterocyclyl having 3 to 9 ring atoms which is optionally substituted by —OH or -Hal).

2. The compound according to claim 1, wherein the hydrocarbon group which contains from 5 to 20 carbon atoms and optionally 1 to 4 heteroatoms selected from O, N and S is selected from 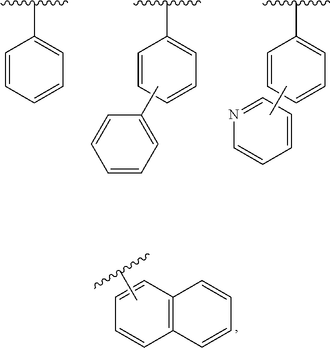
wherein the hydrocarbon group can be optionally substituted as defined in claim 1.

3. The compound according to claim 1, wherein the hydrocarbon group which contains from 5 to 20 carbon atoms and optionally 1 to 4 heteroatoms selected from O, N and S is a monocyclic or polycyclic heterocyclic group having 5 to 15 carbon atoms and 1 or 2 heteroatoms selected from O, N and S,
wherein the hydrocarbon group can be optionally substituted as defined in claim 1.

4. The compound according to claim 1, wherein the viral disease is caused by Herpesviridae, Retroviridae, Filoviridae, Paramyxoviridae, Rhabdoviridae, Orthomyxoviridae, Bunyaviridae, Arenaviridae, Coronaviridae, Picornaviridae, Togaviridae, or Flaviviridae; more specifically wherein the viral disease is influenza.

5. The compound according to claim 1, wherein at least one further medicament which is selected from the group consisting of a polymerase inhibitor which is different from the compound having the general formula (I); neuramidase inhibitor; M2 channel inhibitor;
alpha glucosidase inhibitor; ligand of another influenza target; antibiotics, anti-inflammatory agents, lipoxygenase inhibitors, EP ligands, bradykinin ligands, and cannabinoid ligands is administered concurrently with, sequentially with or separately from the compound having the general formula (I).

6. The compound according to claim 1, wherein the compound having the general formula (I) exhibits an IC₅₀ of less than about 50 μM in the CPE and/or CapFP-LD assay disclosed herein.

7. A method of treating, ameliorating or preventing a viral disease, the method comprising administering to a patient in need thereof an effective amount of a compound having the general formula (I), optionally in the form of a pharmaceutically acceptable salt, solvate, polymorph, prodrug, tautomer, racemate, codrug, cocrystal, enantiomer, or diastereomer or mixture thereof: 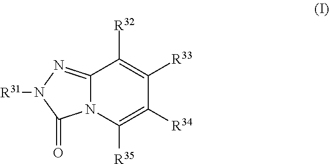
wherein
R³¹ is selected from —H and —C₁₋₆ alkyl;
R³² is selected from —H and —C₁₋₆ alkyl;
R³³ is selected from —H and —C₁₋₆ alkyl;
R³⁴ is selected from —(X³²)ₜ (optionally substituted hydrocarbon group which contains from 5 to 20 carbon atoms and optionally 1 to 4 heteroatoms selected from O, N and S);
R³⁵ is selected from —H and —C₁₋₆ alkyl;
R³⁶ is selected from —H, (optionally substituted C₁₋₆ alkyl), (optionally substituted C₃₋₉ carbocyclyl), —C₁₋₄ alkyl(optionally substituted C₃₋₉ carbocyclyl), (optionally substituted heterocyclyl having 3 to 9 ring atoms), and —C₁₋₄ alkyl (optionally substituted heterocyclyl having 3 to 9 ring atoms);
R³⁸ is selected from —H, (optionally substituted C₁₋₆ alkyl), (optionally substituted C₃₋₉ carbocyclyl), —C₁₋₄ alkyl(optionally substituted C₃₋₉ carbocyclyl), (optionally substituted heterocyclyl having 3 to 9 ring atoms), and —C₁₋₄ alkyl (optionally substituted heterocyclyl having 3 to 9 ring atoms);
X³² is selected from NR³⁶, N(R³⁶)C(O), C(O)NR³⁶, O, C(O), C(O)O, OC(O), N(R³⁶)SO₂, SO₂N(R³⁶), S, SO, and SO₂;
s is 0 to 4; and
t is 0 or 1;
wherein the alkyl group can be optionally substituted with one or more substituents which are independently selected from -halogen, —CN, —CF₃, —OCF₃, —(CH₂)ₛ—X³²—R³⁸, —C₃₋₉ carbocyclyl, and (heterocyclyl having 3 to 9 ring atoms); and
wherein the hydrocarbon group, heterocyclyl group and/or carbocyclyl group can be optionally substituted with one or more substituents which are independently selected from -halogen, —CN, —CF₃, —OCF₃, —(CH₂)ₛ—X³²—R³⁸, —C₁₋₆ alkyl, —C₃₋₉ carbocyclyl which is optionally substituted by —OH or -Hal, —C₁₋₄ alkyl-C₃₋₉ carbocyclyl which is optionally substituted by —OH or -Hal, (heterocyclyl having 3 to 9 ring atoms which is optionally substituted by —OH or -Hal), and —C₁₋₄ alkyl-(heterocyclyl having 3 to 9 ring atoms which is optionally substituted by —OH or -Hal).

8. The method according to claim 7, wherein the hydrocarbon group which contains from 5 to 20 carbon atoms and optionally 1 to 4 heteroatoms selected from O, N and S is selected from 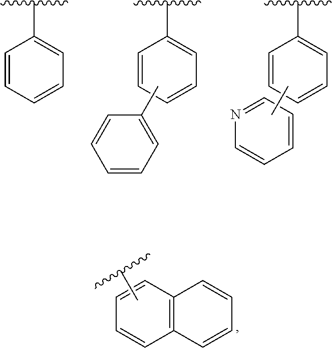
wherein the hydrocarbon group can be optionally substituted as defined in claim 1.

9. The method according to claim 7, wherein the hydrocarbon group which contains from 5 to 20 carbon atoms and optionally 1 to 4 heteroatoms selected from O, N and S is a monocyclic or polycyclic heterocyclic group having 5 to 15 carbon atoms and 1 or 2 heteroatoms selected from O, N and S,
wherein the hydrocarbon group can be optionally substituted as defined in claim 1.

10. The method according to claim 7, wherein the viral disease is caused by Herpesviridae, Retroviridae, Filoviridae, Paramyxoviridae, Rhabdoviridae, Orthomyxoviridae, Bunyaviridae, Arenaviridae, Coronaviridae, Picornaviridae, Togaviridae, or Flaviviridae; more specifically wherein the viral disease is influenza.

11. The method according to claim 7, wherein at least one further medicament which is selected from the group consisting of a polymerase inhibitor which is different from the compound having the general formula (I); neuramidase inhibitor; M2 channel inhibitor; alpha glucosidase inhibitor; ligand of another influenza target; antibiotics, anti-inflammatory agents, lipoxygenase inhibitors, EP ligands, bradykinin ligands, and cannabinoid ligands is administered concurrently with, sequentially with or separately from the compound having the general formula (I).

12. The method according to claim 7, wherein the compound having the general formula (I) exhibits an IC₅₀ of less than about 50 μM in the CPE and/or CapFP-LD assay disclosed herein.
